(19) United States
(12) Patent Application Publication
(10) Pub. No.: US 2017/0049503 A1
(43) Pub. Date: Feb. 23, 2017

(54) ELECTROSURGICAL SYSTEM

(71) Applicant: COSMAN MEDICAL, INC., Burlington, MA (US)

(72) Inventors: Eric R. Cosman, Belmont, MA (US); Eric R. Cosman, Belmont, MA (US)

(73) Assignee: COSMAN MEDICAL, INC., Burlington, MA (US)

(21) Appl. No.: 14/279,286

(22) Filed: May 15, 2014

(51) Int. Cl. A61B 18/14

(52) U.S. Cl. CPC A61B 18/1442; A61B 2018/00005

(57) ABSTRACT

A guideblock can be used to align two or more high-frequency-tissue-ablation probes in a body.

### TECHNICAL FIELD

This invention relates generally to the advances in medical systems and procedures for prolonging and improving human life. The present invention relates generally to a system and method for applying energy, particularly high-frequency (HF) energy such as radiofrequency (RF) electrical energy, to a living body. The present invention also relates generally to a system and method for apply energy for the purpose of tissue ablation.

### BACKGROUND

The theory behind and practice of radiofrequency (RF) heat ablation has been known for decades, and a wide range of suitable RF generators and electrodes exists. RF heat ablation is one example of high-frequency tissue ablation, of which microwave (MW) is another example. For example, equipment for causing heat lesions is available from Radionics, Inc., located in Burlington, Mass. A research paper by E. R. Cosman, et al., entitled “Theoretical Aspects of Radio Frequency Lesions in the Dorsal Root Entry Zone,” Neurosurgery, Vol. 15, No. 6, pp. 945-0950 (1984), describes various techniques associated with radio frequency lesions and is hereby incorporated by reference herein in its entirety. Also, research papers by S. N. Goldberg, et al., entitled “Tissue Ablation with Radio Frequency: Effect of Probe Size, Gauge, Duration, and Temperature on Lesion Volume,” Acad. Radiol., Vol. 2, pp. 399-404 (1995), and “Thermal Ablation Therapy for Focal Malignancy,” AJR, Vol. 174, pp. 323-331 (1999), described techniques and considerations relating to tissue ablation with radio frequency energy and are hereby incorporated by reference herein in its entirety. For a given electrode temperature, size of electrode, and time of heating, you can predict reliably ablation size as described in the papers entitled “Theoretical Aspects of Radiofrequency Lesions and the Dorsal Root Entry Zone,” by Cosman, E. R., et al., Neurosurg 15:945-950, 1984, and “Bipolar Radiofrequency Lesion Geometry: Implications for Palisade Treatment of Sacroiliac Joint Pain.” by E. R. Cosman Jr and C. D. Gonzalez, Pain Practice 2011; 11(1): 3-22 (hereinafter “Cosman and Gonzalez”), which are herein incorporated by reference in their entireties. Examples of high frequency generators and electrodes are given in the papers of entitled “Theoretical Aspects of Radiofrequency Lesions and the Dorsal Root Entry Zone,” by Cosman, E. R., et al., Neurosurg 15:945-950, 1984; and “Methods of Making Nervous System Lesions,” by Cosman, E. R. and Cosman, B. J. in Wilkins R. H., Rengachary S. S. (eds): Neurosurgery, New York, McGraw-Hill, Vol. III, pp. 2490-2498, 1984, and are hereby incorporated by reference herein in their entirety.

United States patents by E. R. Cosman and W. J. Rittman, III, entitled “Cool-Tip Electrode Thermal Surgery System,” U.S. Pat. No. 6,506,189 B1, date of patent Jan. 14, 2003, and “Cluster Ablation Electrode System,” U.S. Pat. No. 6,530,922 B1, date of patent Mar. 11, 2003, described systems and method related to tissue ablation with radiofrequency energy and electrodes and are hereby incorporated by reference herein in their entirety.

The use of radiofrequency (RF) generators and electrodes in neural tissue for the treatment of pain and functional disorders is well known. Included herein by reference, an as an example, the RFG-3C Plus RF Generator of Radionics, Inc., Burlington, Mass., and its associated electrodes are used in the treatment of the nervous system, and the treatment pain and functional disorders. The RFG-3C Plus generator has one electrode output jack for connection to a single active electrode, and it has one reference electrode jack for connection to a reference electrode. When the active electrode is inserted into the body, and the reference electrode is placed, typically on the patient's skin, then RF current form the RF generate flows through the patient's body between the two electrodes. The generator can be activated and its signal output can be applied between the electrodes. Typically, this is referred to as a monopolar configuration because the active electrode is of smaller area than the reference electrode, and so the concentration of RF current is highest near it and the action of the RF electric field, whether for heating or for pulsed RF field therapy is greater there. This usually referred to as a single electrode configuration since there is only one “active” electrode. Parameters that can be measured by the RFG-3C Plus RF generator include impedance, HF voltage, HF current, HF power, and electrode tip temperature. Parameters that may be set by the user include time of energy delivery, desired electrode temperature, stimulation frequencies and durations, and level of stimulation output. In general, electrode temperature is a parameter that may be controlled by the regulation of high frequency output power. Existing RF generators have interfaces that allow the selection of one or more of these treatment parameters, as well as various methods to display the parameters mentioned above.

In another example, the reference electrode can be inserted into the patient's body, and it can have an active area that is smaller and of comparable size to the active electrode. In that case, both electrodes become “active” in the sense that both of the electrodes have high temperature or electrical field effects on the tissues around them, so that they are both involved actively in the therapeutic effects the RF signal output. This can be referred to as a single “bipolar configuration”. Generation bipolar RF lesions is described in a paper by E. R. Cosman, et al., entitled “Radio frequency lesion generation and its effect on tissue impedance”, Appl Neurophysiol 1988; 51:230-242, which is hereby incorporated by reference herein in its entirety. Generation bipolar RF lesions is described in U.S. Pat. No. 5,433,739 by E. R. Cosman and M. E. Sluijter, which is hereby incorporated by reference herein in its entirety. Generation bipolar RF lesions using cooled and non-cooled RF electrodes is described in U.S. patent application Ser. No. 08/562,986 by W. J. Rittman III and E. R. Cosman, filed Nov. 24, 1995, which is hereby incorporated by reference herein in its entirety. Generation bipolar RF lesions is described in a paper by M. F. Ferrante, et al., entitled “Radiofrequency Sacroiliac Joint Denervation for Sacroiliac Syndrome”, Reg Anesth Pain Med 2001; 26(2):137-142, which is hereby incorporated by reference herein in its entirety. Generation bipolar RF lesions is described in a paper by C. A. Pino, et al., entitled “Morphologic Analysis of Bipolar Radiofrequency Lesions: Implications for Treatment of the Sacroiliac Joint”, Reg Anesth Pain Med 2005; 30(4):335-338, which is hereby incorporated by reference herein in its entirety. Generation bipolar RF lesions is also described in a paper by R. S. Burnham, et al., entitled “An Alternate Method of Radiofrequency Neurotomy of the Sacroiliac Joint: A Pilot Study of the Effect on Pain, Function, and Satisfaction”, Reg Anesth Pain Med 2007; 32(1):12-19, which is hereby incorporated by reference herein in its entirety. Generation of bipolar lesions for SIJ deneravation, spinal pain management, tumor ablation, and surgical tissue devasculatization of bodily organs, including generation of bipolar lesions using a guideblock, is described in U.S. application Ser. No. 13/081,873 filed on Apr. 7, 2011, which incorporated by reference in its entirety. Generation of multiple bipolar lesions with temperature control for each electrode is described in U.S. application Ser. No. 12/835,489 filed on Jul. 13, 2010, which incorporated by reference in its entirety. Generation of bipolar RF heat lesions is described in “Bipolar Radiofrequency Lesion Geometry: Implications for Palisade Treatment of Sacroiliac Joint Pain.” by E. R. Cosman Jr and C. D. Gonzalez, Pain Practice 2011; 11(1): 3-22 (hereinafter “Cosman and Gonzalez”), which are herein incorporated by reference in their entireties. Cosman and Gonzalez describe the method of using fluoroscopic x-ray guidance to place a series of RF cannula along a path lateral to the lateral aspect of the sacral foramina and medial to the sacroiliac joint (SIJ) line, and creating a series of bipolar RF heat lesions between adjacent pairs of cannulae to create an extended lesion zone that traverses the space through which the sacral lateral branch nerves are known to travel, but travel at irregular locations without regular reference to bony landmarks. RF cannula and electrode used in pain management typically include elongated stainless-steel shafts that are visible in x-ray imaging. Fluoroscopic (x-ray) imaging is a common means of image-guidance for pain management procedures in and around the spine, and in targeting peripheral nerves throughout the body where nerves have regular relationship to bony landmarks, because bone are visible in x-ray images, but soft tissue (eg nerves) are generally not. One limitation of the prior art is a guideblock comprising markers visible in x-ray imaging is not used for placement of one or more pairs of bipolar high-frequency ablation electrodes.

Four patents have issued on pulsed radiofrequency (PRF) by Sluijter M. E., Rittman W. J., and Cosman E. R. They are “Method and Apparatus for Altering Neural Tissue Function,” U.S. Pat. No. 5,983,141, issued Nov. 9, 1999; “Method and System for Neural Tissue Modification,” U.S. Pat. No. 6,161,048, issued Dec. 12, 2000; “Modulated High Frequency Tissue Modification,” U.S. Pat. No. 6,246,912 B1, issued Jun. 12, 2001; and “Method and Apparatus for Altering Neural Tissue Function,” U.S. Pat. No. 6,259,952 B1, issued Jul. 10, 2001. These four patents are hereby incorporated by reference herein in their entirety. PRF is one example of a high-frequency electrosurgical method. PRF can be used in both monopolar and bipolar configurations. The alignment of electrodes energized in PRF configuration, such as a bipolar configuration, influences the geometry of the PRF electric field.

The use of high frequency electrodes for heat ablation treatment of functional disease and in the destruction of tumors is well known. One example is the destruction of cancerous tumors of the kidney using radio frequency (RF) heat ablation. A paper by D. W. Gervais, et al., entitled “Radio Frequency Ablation of Renal Cell Carcinoma: Early Clinical Experience,” Radiology, Vol. 217, No. 2, pp. 665-672 (2000), describes using a rigid tissue perforating and penetrating electrode that has a sharpened tip to self-penetrate the skin and tissue of the patient. This paper is hereby incorporated by reference herein in its entirety. A paper by Luigi Solbiati et al. entitled “Hepatic Metastases: Percutaneous Radiofrequency Ablation with Cool-Tip Electrodes,” Radiology 1997, vol. 205, no. 2, pp. 367-373 describes various techniques and considerations relating to tissue ablation with RF electrodes which are internally-cooled by circulating fluid, and is incorporated herein by reference. A paper by Rosenthal et al entitled “Percutaneous Radiofrequency Treatment of Osteoid Osteoma,” Seminars in Musculoskeletal Radiology, Vol. 1, No. 2, 1997 reports the treatment of a primary benign bone tumor and the management of concomitant pain using a percutaneously placed radiofrequency electrode, and is incorporated herein by reference.

The present invention overcomes the stated disadvantages and other limitations of the prior art.

### SUMMARY OF THE INVENTION

In one aspect, the present invention is directed toward the problem of aligning high-frequency ablation electrodes within the body, wherein the electrodes can be energized in monopolar, bipolar, or multipolar configurations. In one aspect, the present invention is directed toward the problem of radiofrequency electrodes/cannulae, including both cooled RF and non-cooled RF electrode systems, in the living body for radiofrequency heat lesioning and pulsed radiofrequency (PRF) heat lesioning procedures wherein electrodes are energized in monopolar, bipolar, multipolar, and simultaneous, rapidly switching, and sequential combinations thereof. In one aspect, the present invention is directed toward the problem of aligning a guideblock to anatomy for bipolar RF lesioning between two physically separate RF electrodes/cannulae.

In one aspect, the present invention is related to the problem of aligning to two or ablation probes in a geometric configuration in a body to produce a heat lesion zone of a desired geometry in a target region of the body. Alignment can include constraining the relative orientation of two or more ablation probes, the relative position of two or more probes, or both relative orientation and position of two or more ablation probes.

In one aspect, the present invention is related to the problem of aligning the active tips of two or more ablation probes sufficiently nearby each other, and with desirable relative positions and orientations (such as parallel), at a depth within bodily tissue to produce a single ablation volume (which can be referred to as a cluster ablation), for example by energizing all probes which the same electrical ablation signal or with electrical ablation signals that differ in amplitude, timing, or other characteristics across the ablation probes.

In one aspect, the present invention is related to the problem of aligning the active tips of two or more ablation probes in a parallel configuration at a depth within bodily tissue to enhance bipolar radiofrequency heating between the active tips of pairs of the ablation probes. In one aspect, the present invention is related to the problem of aligning the active tips of two or more ablation probes in a row wherein all active tips are substantially parallel to each other at a depth within bodily tissue to enhance bipolar radiofrequency heating between the active tips of adjacent pairs of the ablation probes, for example for heating of the sacral lateral branch nerves between the lateral aspect of the dorsal sacral foramina and the sacroiliac joint line.

In one aspect, the present invention is related to the use of image guidance in the use of guideblocks for alignment of two or more ablation probes in a body. In one aspect, the present invention is related to problem of aligning a guideblock to bodily structures to facilitate placement of two or more ablation probes at desired locations and orientation, and to avoid anatomical constraints in the placement of ablation probes such as those due to the need to avoid sensitive structure and/or structures through which some ablation probes cannot physically pass, such as hard bone.

In one aspect, the present invention relates to a guideblock for alignment of two or more high-frequency-tissue-ablation probes in a body.

In one aspect, the present invention relates to a guideblock that aligns two or more ablation probes in a body, in one or more of the configurations selected from the group: all probes parallel, no two probes are parallel, some probes are parallel and some probes are not parallel, probes spaced for bipolar ablation, probes spaced for the production of overlapping bipolar lesions, probes spaced for bipolar RF ablation, probes spaced for bipolar RF ablation between the active tips of physically separate probes, probes spaced for generation of a singular bipolar RF lesion that fills in the space between two physically separate ablation probes, probes spaced for monopolar ablation, probes spaced for the generation of overlapping monopolar lesions, probes spaced for monopolar RF ablation, probes spaced for microwave ablation, probes spaced to form a cluster, probes oriented to avoid an anatomical structure, probes positioned to form a cluster within a target a first anatomical region or regions and to avoid an second anatomical region or regions, probes positioned to avoid one or more ribs and to position the probe active tips in a desired arrangement in a target anatomical region, probes positioned to avoid one or more ribs and to position the probe active tips in a cluster in the liver, probes positioned to avoid one or more ribs and to position the probe active tips in a cluster in the lung, probes positioned perpendicular to a bone surface, probes positioned parallel to a bone surface, proves positioned tangent to an anatomical surface, probes positions perpendicular to an anatomical surface, probes positioned in a row, probes positioned in a straight row, probes positioned in a curved row, probes positioned in a triangular array, probes positioned in an isolosceles triangle arrangement, probes positioned in a square array, probes positioned in a rectangular array, probes positioned in a parallel polygonal array, probes positioned in two parallel rows, probes positioned such that a first probe and second probe are parallel and third probe is not parallel to the first probe or second probe, probes perpendicular to the dorsal sacrum, probes positioned with distal points touching or almost touching the dorsal sacrum, probes positioned across space between the transverse processes of two adjacent thoracic vertebra, probes positioned perpendicular to the lateral articular pillar of a cervical vertebra, probes positioned tangent to the lateral articular pillar of a cervical vertebra, probes positioned tangent to the notch between the transverse process and the superior articular process of a lumbar vertebra, probes positioned perpendicular to the notch between the transverse process and the superior articular process of a lumbar vertebra, probes positioned in an intervertebral disc, probes positioned in the posterior annulus of an intervertebral disc, probes positioned along a the path of a desired cut, probes positioned in an organ in a desired arrangement, probes positions in a bone in a desired arrangement, probes positioned in a vertebra, probes positioned across the basivertebral nerve in a vertebra, probes position within or across an osteoid osteoma.

In one aspect, the present invention relates to a guideblock that aligns two or more ablation probes in a body, wherein the guideblock includes one or more markers for image guidance of guideblock position relative to the body, including but not limited to image guidance by means of markers visible relative to body anatomy in x-ray, fluoroscopy x-ray, CT, MRI, PET images.

In one aspect the present invention relation to a guideblock includes slots configured to aligns two or more ablation probes in a body, wherein the guideblock includes a medical-image-visible marker or markers that uniquely identifies each ablation-probe slot of the guideblock in a medical image.

In one aspect, the present invention relates to a guideblock that aligns two or more ablation probes in a body in a configuration wherein one or more ablation probes is either not parallel to another ablation probe or to a principle direction of a medical image including the guideblock, the guideblock including a marker that indicates the position in the body of the active tip of an ablation probe inserted to a predetermined depth in the body using the guideblock.

In one aspect, the present invention relates to a guideblock that aligns two or more ablation probes in a body and a depth stop that fixes the depth of insertion of an ablation probe using the guideblock.

The present invention relates to the use of guideblock with ablation probes including but not limited to ablation probes of the type included in the group: radiofrequency cannula, radiofrequency electrode, radiofrequency injection electrode, radiofrequency electrode without completely uninsulated shaft, RF probe whose shaft includes an active tip and an electrically-insulated region that does not conduct RF current to tissue, monopolar radiofrequency, bipolar radiofrequency, multi-contact radiofrequency electrode, cooled RF, impedance-controlled cooled RF, temperature-controlled cooled RF, cooled RF electrode with extension tip thermosensor, cooled radiofrequency electrode with movable thermosensor positioned distal to the active tip, radiofrequency electrode controlled using a satellite thermosensor that is physically separate from the radiofrequency electrode, cooled radiofrequency electrode controlled using a satellite thermosensor that is physically separate from the radiofrequency electrode, internally-cooled radiofrequency electrode, fluid-cooled RF electrode, perfusion radiofrequency electrode, bipolar cooled RF, internally-cooled electrode, perfusion electrode, microwave, microwave antenna, internally-cooled microwave electrode, direct current, high-frequency current, high-frequent stimulation nerve block signal, stimulation, neurolytic injection needle, injection needles, anesthetic injection needle.

In one aspect, the present invention relates to sizing a guideblock for alignment of two or more ablation probes to stabilize the portion of the ablation probe that is outside a body, for example in the cases where the length of the ablation probes is longer than the depth of insertion into the body.

The present invention relates to the use of guideblocks for alignment of two or more ablation probes for clinical objectives including but not limited to tumor ablation, partial tumor ablation, tissue ablation, tissue coagulation, tissue devascularization, tissue devascularization for bloodless resection, an open surgical procedure, percutaneous tissue ablation, laparocopic ablation nerve ablation, brain ablation, ablation of the intervertebral disc, ablation of a bone, ablation of a vertebra, ablation of osteoid osteoma, pain management, cancer therapy, movement disorder treatment, neurosurgery, surgical tumor resection, surgical tissue resection, ablation in the liver ablation, ablation in the lung, ablation in the pancreas, ablation in the kidney, ablation in the adrenal gland, ablation in the thyroid, SIJ denervation, ablation of some or all of the dorsal sacral innervation, ablation of the sacral lateral branch nerves, ablation of the lumbar L5 dorsal ramus nerve or branch thereof, ablation of a lumbar medial branch nerve, ablation of a thoracic medial branch nerve, ablation of a cervical medial branch nerve, facet denervation, ablation of a peripheral nerve, ablation of the sympathetic chain.

In one aspect, the present invention relates to a guideblock that aligns two or more ablation probes in a body and that includes a multiplicity of slots that provide for physician selection of the relative position of ablation probes.

In one aspect, the present invention relates to a guideblock that aligns two or more ablation probes in a body and that includes a multiplicity of slots that provide for physician selection of the relative position of ablation probes, and indicators of the relative spacing of the slots, such as an intergrated ruler, numerical markings, tick marks, and other indicators of distance or angle.

In one aspect, the present invention relates to a guideblock that aligns two or more ablation probes in a body by means of slots with lateral openings that provide for the lateral separation of the guideblock and ablation probes inserted through the slots.

In one aspect, the present invention relates to a guideblock system that aligns two or more ablation probes in a body by means of slots with lateral openings that provide for the lateral separation of the guideblock and ablation probes inserted through the slots, wherein the guideblock system further includes a clamp for closing off one or more of the lateral openings.

In one aspect, the present invention relates to a guideblock system that aligns two or more ablation probes in a body by means of slots, and that includes a clamp for locking an ablation probe into a slot.

In one aspect, the present invention relates to a guideblock system that positions an ablation probe and a physically separate temperature sensor relative to each other in a body, and an electrosurgical generator that adjusts the ablation signal output delivered to the ablation probe in whole or in part by a temperature measured by physically separate temperature sensor.

In one aspect, the present invention relates to a guideblock including a row of slots for placement of a row of radiofrequency ablation probes at or near the dorsal surface of the sacrum, wherein the slots orient the radiofrequency ablation probes such that they are parallel to each other, and the slots space the probes for generation of bipolar radiofrequency heat lesions between each adjacent pair of radiofrequency ablation probes.

In one aspect, the present invention relates to a guideblock including three slots for alignment of three internally-cooled RF electrodes around the lateral aspect of a sacral foramina.

In one aspect, the present invention relates to a method for tissue ablation comprising orienting a guideblock to a body, inserting two or more ablation probes into the body through the guideblock, ablating tissue using the ablation probes.

In one aspect, the present invention relates to a method of placement of two or more ablation probes in a geometric arrangement within the living body including imaging a guideblock in relation to a body, determining a target location in the body for each ablation probe by means of the image of a marker visible in the image and having a geometric relationship to slots in the guideblock, inserting each ablation probe through a slot in the guideblock into the body, ablating tissue using the ablation probes.

In one aspect, the present invention relates to a method of placement of two or more ablation probes in a geometric arrangement within the living body including imaging a guideblock in relation to a body, determining a target location in the body for each ablation probe by means of the image of a marker visible in the image and having a geometric relationship to slots in the guideblock, inserting each ablation probe through a slot in the guideblock into the body, ablating tissue using the ablation probes, and then removing all ablation probes except one remaining ablation probe, repositioning the guideblock relative to the one remaining ablation probe, re-inserting one or more ablation probes through a slot in the guideblock into the body, and ablating tissue using the ablation probes.

The invention can be used in numerous organs in the body, including the brain, spine, liver, lung, bone, kidney, abdominal structures, etc., and for the treatment of cancerous tumors, other pathological target volumes, or other types of tissue target volumes in, for example, nervous tissue, a nerve located within a bone, bone tissue, cardiac tissue, muscle tissue, or other types of bodily tissues.

Other examples of embodiments of systems and methods of the present invention are given in the rest of this patent. The details of embodiments of the invention are set forth in the accompanying drawings and description below. Other features, objects, and advantages of the invention will be apparent from the description and drawings and from the claims.

### BRIEF DESCRIPTION OF THE DRAWINGS

In the drawings that constitute a part of the specification, embodiments exhibited various forms and features hereof are set forth, specifically:

FIG. 1A is a schematic diagram showing a guideblock for alignment of high-frequency ablation electrodes including radiopaque markers used to align the guideblock to anatomy lumbar and sacral spine using fluoroscopic x-ray imaging.

FIG. 1B is a schematic diagram showing, in a lateral view, the guideblock used to position radiofrequency ablation probes to perform bipolar radiofrequency ablation of the dorsal innervation of a sacroiliac joint.

FIG. 1C is a schematic diagram showing, in a posterior view, the guideblock used to position radiofrequency ablation probes to perform bipolar radiofrequency ablation of the dorsal innervation of a sacroiliac joint.

FIG. 1D is a schematic diagram showing, in three perpendicular views, the guideblock including x-ray visible markers.

FIG. 2 is a schematic diagram showing, in three perpendicular views, a guideblock including x-ray-visible markers, and having unequal spacing between adjacent holes for alignment of ablation electrodes.

FIG. 3 is a schematic diagram showing, in three perpendicular views, a guideblock including an x-ray visible marker for each ablation-probe guide hole.

FIG. 4 is a schematic diagram showing, in three perpendicular views, a guideblock wherein the guide channel for each high-frequency ablation electrode includes a x-ray visible material.

FIG. 5 is a schematic diagram showing, in three perpendicular views, a guideblock including a guide-hole and a radiopaque marker for each of two ablation probes.

FIG. 6 is a schematic diagram showing, in three perpendicular views, a guideblock with surface contoured to match the anatomical surface on which the the guideblock configured to be positioned.

FIG. 7 is a schematic diagram showing, in three perpendicular views, a guideblock having weight-reducing cross-section, a laterally-supportive base, and two x-ray-visible markers flanking the guide-slot for each ablation probe.

FIG. 8 is a schematic diagram showing, in three perpendicular views, a guideblock for aligning ablation probes in a non-parallel configuration, and having an x-ray-visible marker for each ablation probe that indicates the position of the distal end of the ablation probe when the probe is inserted into the body at a depth.

FIG. 9A is a schematic diagram showing, in three perpendicular views, a guideblock for aligning ablation probes including guide-hole with lateral slots that provide for adjustment of individual ablation-probe location and for removal of the guideblock when the ablation probes are inserted into a living body through the guideblock.

FIG. 9B is a schematic diagram showing, in three perpendicular views, a guideblock for aligning ablation probes including guide-hole with lateral slots that provide for adjustment of individual ablation-probe location and for removal of the guideblock when the ablation probes are inserted into a living body through the guideblock, additionally including a clamping plate for securing the ablation probe in the guide-holes.

FIG. 10 is a schematic diagram showing, in three perpendicular views, a guideblock for aligning ablation probes along a curved line.

FIG. 11 is a schematic diagram showing, in three perpendicular views, a guideblock for aligning ablation probes including a common connection between the ablation probes and an electrosurgical generator.

FIG. 12 is a schematic diagram showing, in three perpendicular views, a guideblock for aligning ablation probes in a parallel rectangular array.

FIG. 13 is a schematic diagram showing, in two perpendicular views, a guideblock for aligning three ablation probes in a variety of parallel triangular arrangements.

FIGS. 14A-C are a schematic diagrams showing several views of a guideblock for aligning three ablation probes in a variety of triangular arrangements in which two probes are parallel and the third probe is not parallel to the other two probes.

FIG. 15A is a schematic diagram showing, in three perpendicular views, a guideblock for aligning three ablation probes equidistant from a central location.

FIG. 15B is a schematic diagram showing, in a posterior view, two guideblocks, each aligning three ablation probes from the lateral aspect of a sacral foramina.

### DETAILED DESCRIPTION OF THE INVENTION

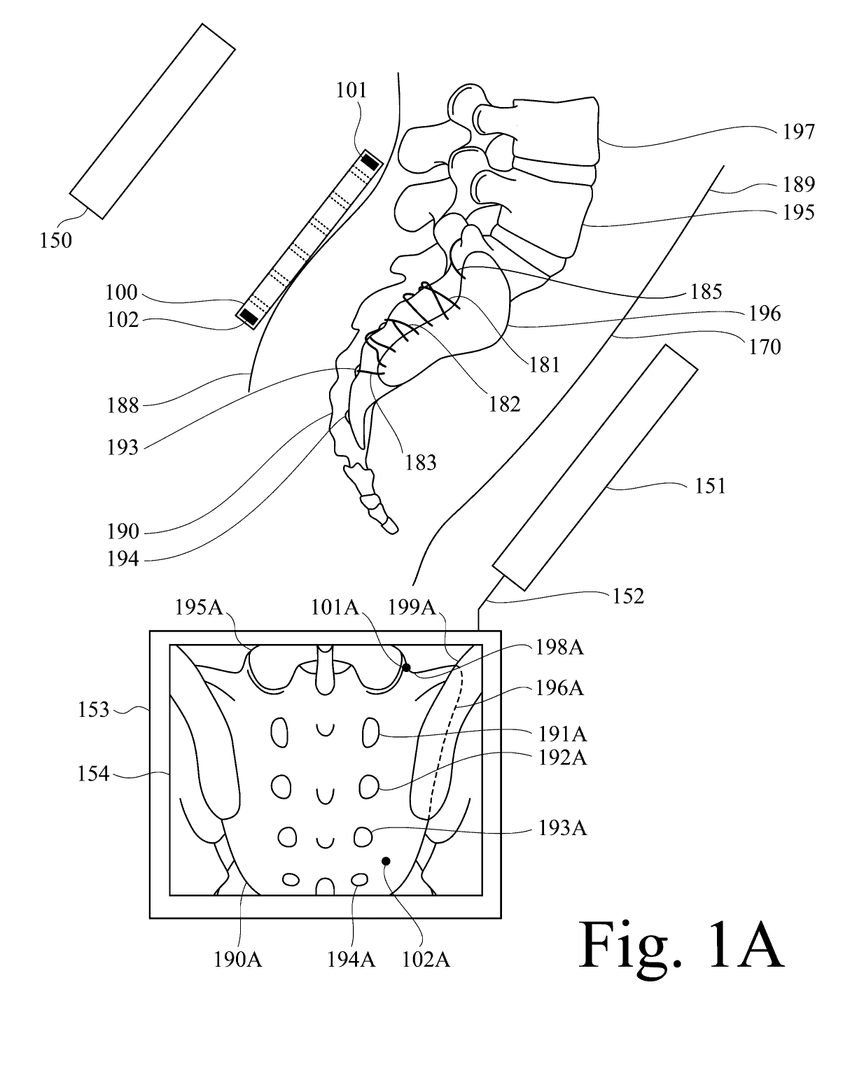

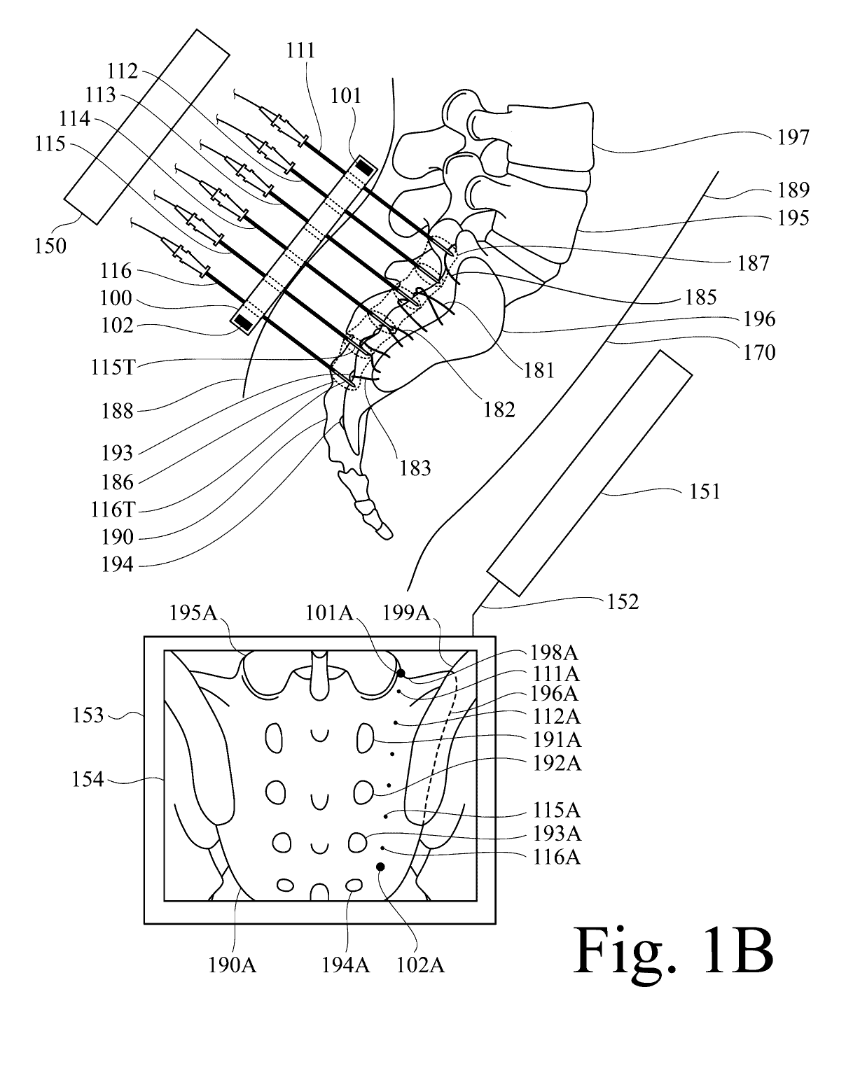

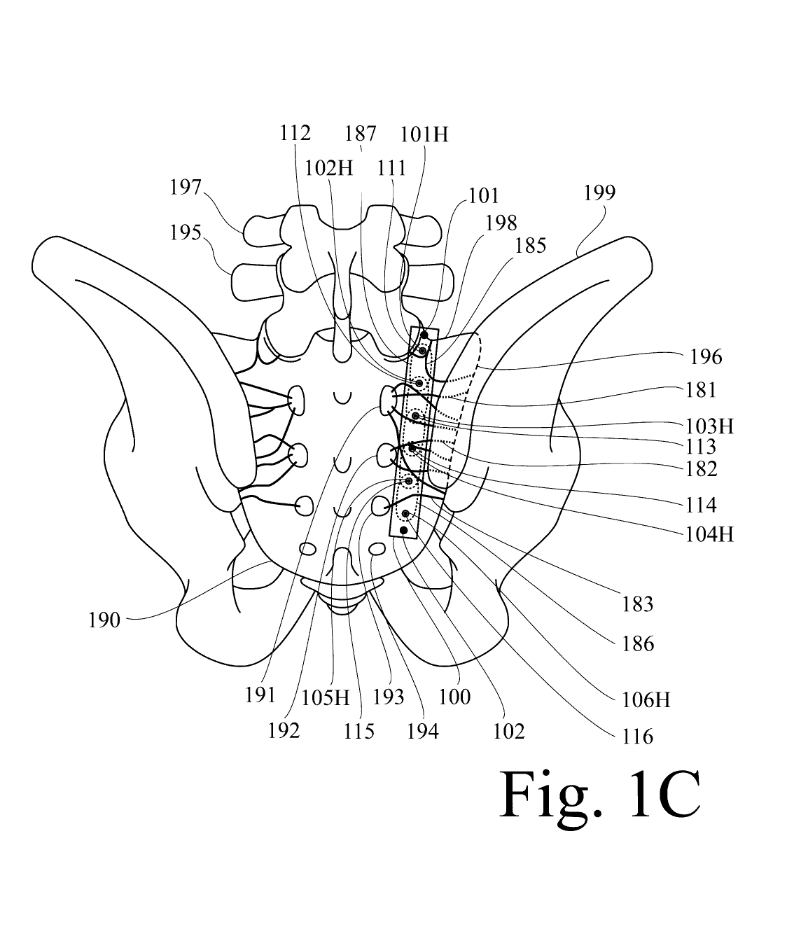

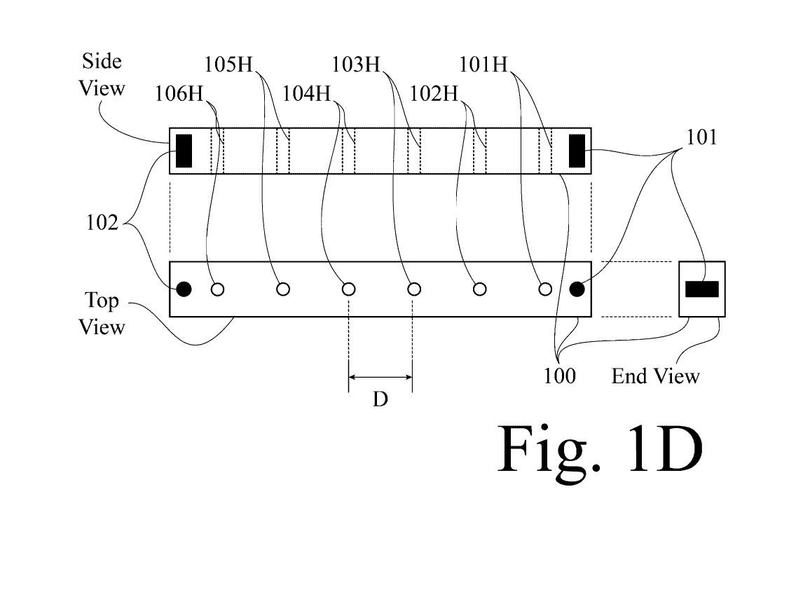

Referring now to FIG. 1, in accordance with several aspects of the present invention, FIG. 1 refers collectively to FIG. 1A, FIG. 1B, FIG. 1C, and FIG. 1D. FIG. 1 presents schematically several embodiments of an apparatus for high-frequency ablation of tissue within a patient 170, in accordance with the present invention. FIG. 1 shows a guideblock 100 with x-ray visible markers 101 and 102; an x-ray imaging system comprising an x-ray-source 150, x-ray detector 151, x-ray image viewer 153, and x-ray image 154 displaying marker images 101A and 102A in relation to the image of x-ray-visible parts of the target anatomy 190A, 195A, 199A of body 170. FIGS. 1B and 1C additionally show HF ablation probes 111, 112, 113, 114, 115, 116 aligned by guideblock 100, inserted into body 170 and applying signal output from high-frequency generator (eg RF or MW) to body 170. In one aspect, FIG. 1 shows a method of ablation of the dorsal innervation of the SU comprising using fluoroscopic x-ray guidance to place a guideblock including x-ray-visible markers relative to target anatomy; using the guideblock and x-ray guidance to place a series of RF cannula along a path lateral to the lateral aspect of the sacral foramina and medial to the sacroiliac joint (SU) line; and creating a series of bipolar RF heat lesions between adjacent pairs of cannulae to create an extended lesion zone that traverses the space through which the sacral lateral branch nerves and/or branches of the L5 spinal nerve are known to travel, but travel at irregular locations without regular reference to bony landmarks.

In FIG. 1, the target anatomy includes the sacral lateral branch nerves originating from S1 sacral foramina 191, S2 sacral foramina 192, S3 sacral foramina 193 (and sometime S4 sacral foramina 194) and innervating the sacroiliac joint (SIJ) 196. The target anatomy also includes a branch 185 of the L5 dorsal ramus crosses the sacral ala 198 innervates the SU 196 and the L5-S1 facet joint. Nerves 181, 182, 183 are each one sacral lateral branch nerve originating from S1, S2, S3 sacral foramina, respectively, and are positioned over the dorsal surface of the sacrum 190. The L5 vertebral 194 and L4 vertebra 197 are part of the lumbar spine. Guideblock 100 is positioned at or over the dorsal skin surface 188. The x-ray apparatus 150, 151, 153 can be a fluoroscopic C-arm imagine device or a film x-ray machine, as is familiar to one skilled in the art of x-ray imaging. In the case the x-ray apparatus is a fluoroscopy device, connection 152 is a data connection that transfers imaging data from the image intensifier 151 to the display 153 of a fluoroscopy imaging device. The x-ray source 150 is positioned posterior to the posterior body 188 and the image intensifier (or film holder) 151 is positioned anterior to the anterior skin of the body 189 to produce a posterior-anterior (PA) image 154 shown on display (or film) 153.

In FIG. 1A, in lateral view of the anatomy and guideblock, the guideblock 100 is aligned to the soft-tissue anatomy 181, 182, 183, 185 using x-ray image 153 of the guideblock radiopaque markers 101, 102 and bony anatomy 190, 199, 195 which has known physical relationship to the target nerves. In x-ray image 153, the markers 101 and 102 appear as images 101A and 102A, respectively. In x-ray image 153, the sacrum 190 appears as image 190A, the illium 199 appears as image 199A, the sacral ala 198 appears as image 198A, the L5 verebra appears as image 195A, the SIJ 196 appears as image 196A, the S1 foramina 191 appears as image 191A, the S2 foramina 192 appears as image 192A, the S3 foramina 193 appears as image 193A, the S4 foramina 194 appears as image 194A. The position and orientation of the guideblock 100 can be adjusted before an ablation probe 111 (that may itself be visible in x-ray) is introduced into the body, by observation of the position of marker images 101A and 101B relative to images of the target anatomy, including, but not limited to, the lateral aspect of the sacral foramina 191A, 192A, 193A, 194A, the SIJ line 196A, the sacral ala 198A, illium 199A, and the sacrum 190A. The two markers 101, 102 can be used to visualize the orientation, lateral position, and superior-inferior position and extent of the line of ablation probes 111, 112, 113, 114, 115, 116 after their insertion through the guideblock 100 and into the body 170, as shown in FIG. 1B and FIG. 1C.

In FIG. 1B showing a lateral view of the anatomy and guideblock, and in FIG. 1C showing a posterior view of the anatomy and guideblock, ablation probes 111, 112, 113, 114, 115, 116 are inserted through guideblock 100, through the skin 188, and into contact with the dorsal sacrum 190, between the lateral aspect of the sacral foramina and the SIT. The ablation probes each have an enogated shaft with a proximal end and a distal end, the proximal end including a means of connection to an electrodsurgical generator such as a hub and a cable, and the distal end including an active tip, such as tip 115T of probe 115, and tip 116T of probe 116, for delivery of a high-frequency electrical signal to the body 170, which can be the body of a living patient being treated for SIJ pain. In FIG. 1C, the anatomy is depicted as visible through the guideblock 100 and the skin surface 188 for clarity. The ablation probes 111, 112, 113, 114, 115, 116 are visible as images 111A, 112A, 113A, 114A, 115A, 116A in x-ray image 153. The ablation probes 111, 112, 113, 114, 115, 116 are held in a substantially parallel configuration by the guideblock 100, which includes parallel slots 101H, 102H, 103H, 104H, 105H, 106H through which the elongated shaft of probes 111, 112, 113, 114, 115, 116 pass with equal distance D between each adjacent pair of probes. Typically a probe shaft is cylindrical, but generally probe shaft can have other cross-sectional shapes. Each ablation probe is connected to an electrosurgical generator, such as a HF generator such as an RF generator or a MW generator, via a cable which is partially shown in the figure; the generator is not shown for clarity, but can be of a type that is known to one skilled in the art of HF tissue ablation. In the embodiment shown in FIG. 1, a bipolar radiofrequency heat lesion is generated between each pair of adjacent ablation probe by passing electrical current between each adjacent pair of ablation probes. For example, bipolar lesion 186 is formed by passing HF current between the active tips 116T and 115T. For example, bipolar lesion 187 is formed by passing HF current between the active tips of electrodes 111 and 112. The five bipolar heat lesions shown in FIGS. 1B and 1C can be generated one at a time sequentially, two at a time sequentially (for example using the Cosman G4 generator), and all at the same time using the systems and methods presented in U.S. application Ser. No. 12/835,489. The bipolar lesions are positioned and sized to interrupt the sacral lateral branch nerves 181, 182, 183 and a branch of the L5 dorsal ramus 185 that innervates the SIJ and/or the L5-S1 facet joint. The guideblock 100 keeps the shafts of the electrodes parallel and with consistent spacing to ensure heating of the inter-tip region between adjact electrodes, as described in Cosman and Gonzalez (2011). The ablation probes can take the form of one or more RF electrode types selected from the group: an RF cannula (such as the Cosman CC or RFK cannula) including an exposed active tip at its distal end and being energized by a thermocouple-equiped RF electrode (such as the Cosman TCD, CSK, or TCN electrodes), an injection electrode such as the Cosman CU or CR electrode, a solid electrode such as the Cosman RRE electrode, and side-outlet electrode wherein the electrode merges from a lateral apparature in the introducer needle, a single-prong RF electrode, a two-prong RF electrode, a multi-prong RF electrode, an internally-cooled RF electrode, an internally-cooled RF electrode with rounded distal end and thermosensor at the distal end, an internally-cooled RF electrode with thermosensing extension tip, an internally-cooled RF electrode including a microwave radiometer configured to sense tissue temperature several millimeters (eg 2-3 mm) distal to the distal end of the ablation probe, a temperature-sensing RF electrode, a non-temperature-sensing RF electrode. The active tip of each electrode 111, 112, 113, 114, 115, 116 can have a diameter selected from the group: 23, 22, 21, 20, 19, 18, 17, 16, 15, 14 gauge, a diameter smaller than 23 gauge, a diameter larger than 14 gauge. In some embodiment, a system can include a multiplicity of guideblocks, each configured for a different ablation probe diameter, which can be selected by physician to suit clinical needs. The active tip of each electrode 111, 112, 113, 114, 115, 116 can have a length selected from the group: 2, 3, 4, 5, 5.5, 6, 7, 8, 9, 10 mm, between 10 mm and 15 mm inclusive, between 15 mm and 20 mm inclusive, a length shorter than 2 mm, and length longer than 20 mm. The shaft length each electrode 111, 112, 113, 114, 115, 116 can have a length selected form the group: 5 cm, 7.5 cm, 10 cm, 12 cm, 15 cm, 20 cm, 25 cm, a length less than 5 cm, a length between 5 cm and 15 cm, a length longer than 15 cm, a length longer than 25 cm. Each slot 101H, 102H, 103H, 104H, 105H, 106H can have a dimension configured to allow smooth, non-damaging passage of its respective electrode 111, 112, 113, 114, 115, 116, while keeping the electrode substantially parallel to adjacent electrodes. For example, clearance between the outer diameter of the ablation electrode 111 and the inner diameter of the slot 101H can a value selected from the group: 0.001″, 0.002″, 0.003″, 0.004″, 0.001-0.004″, 0.004-0.006″, 0.006″-0.01″, a value less than 0.001″, and value greater than 0.01″. In some embodiments, a guidehole diameter can be only modestly larger than the outer diameter of an ablation probe to constrain the ablation probe direction, and the orientation of the ablation probe relative to another ablation probe or probes. In some other embodiments, a guidehole diameter can be large relative to the outer diameter of an ablation probe to allow the physician flexibility in orienting the ablation probe relative to other ablation probes and anatomy; for example in the case where a physician is positioning RF cannula relation to one or more sacral foramina, different physicians may put different emphasis on the desire to fix the relative spacing of two or more RF cannula, and the desire to adjust the position of an RF cannula that erroneously enter a sacral foramina after other RF cannula have already been placed, and thus providing more than one guideblock with different degrees of tightness in the ablation probe-to-guidehole fit can be desirable. The spacing D between adjacent holes, for example hole 101H and hole 102H, can be a distance selected from the group: 5 mm, 6 mm, 10 mm, 12 mm, 15 mm, 17 mm, 20 mm, 25 mm a value in the range 5-25 mm, a value less than 5 mm, a value greater than 25 mm, a distance configured to produce consistent inter-tip heating for a time setting, temperature setting, active tip length, and the active tip gauge size that guideblock slots 101H, 102H, 103H, 104H, 105H, 106H are configured to guide. In some embodiments, the guideblock 100 can include holes spaced by 10 mm for guidance of 20-gauge cannula. In some embodiments, the guideblock 100 can include holes spaced by 12 mm for guidance of 18-gauge cannula. In some embodiments, the guideblock 100 can include holes spaced by 15 mm for guidance of 16-gauge cannula.

FIG. 1D shows the guideblock 100 in three perpendicular views: a top view, a side view, and a view of the block's end. The block 100 can comprise a plastic block including six parallel through-holes 101H, 102H, 103H, 104H, 105H, 106H, equally spaced by distance D, and each is configured to allow passage of, and to provide alignment for, an ablation probe, such as a radiofrequency electrode. The dotted lines in the Side View labeled 101H, 102H, 103H, 104H, 105H, 106H indicate the through-holes passing through the mass of the guideblock 100. The markers 101 and 102 can be fully encased in the guideblock 100, but visible externally if the guideblock's main structure is composed from a transparent or translucent material. The block 100 can include a material selected from the group: polypropylene, ABS, delrin, polycarbonate, acrylic, a hard plastic, a moldable plastic, an injection moldable plastic, a machinable plastic. In some embodiments, the guideblock comprises an injection-molded part or parts. In some embodiments, the guideblock comprises a machined part or parts. In a preferred embodiment, the block 100 can be radiolucent except for the markers 101 and 102. In some embodiments, the block 100 can be substantially invisible in x-ray images except for the markers 101 and 102. In some embodiments, the block 100 can include radiopaque elements. In some embodiments, the block 100 can be completely radiopaque, composed of a substance such as stainless steel. The block 100 can be a single solid piece into which x-ray markers are inserted and holes are drilled. The guideblock 100 can be single-use and provided sterile. The guideblock 100 can be multi-use and sterilizable. The x-ray markers 101 and 102 are collinear with the row of slots 101H, 102H, 103H, 104H, 105H, 106H, indicating the orientation and extent of the row of slots. The x-ray markers 101 and 102 can include an x-ray-visible material such as platinum, gold, stainless steel, tantalum, metal-impregnated plastics, tungsten-filled polymers, tungsten-filled nylon, tungsten-filled urethane, tungsten-filled thermoplastic elastomers or other materials known visible in x-ray images. The markers 101 and 102 can have a elongated extent in the direction of the slots 101H, 102H, 103H, 104H, 105H, 106H so that the user can ascertain the alignment of the block to the x-ray beam by the extent and/or shape of each marker's x-ray image.

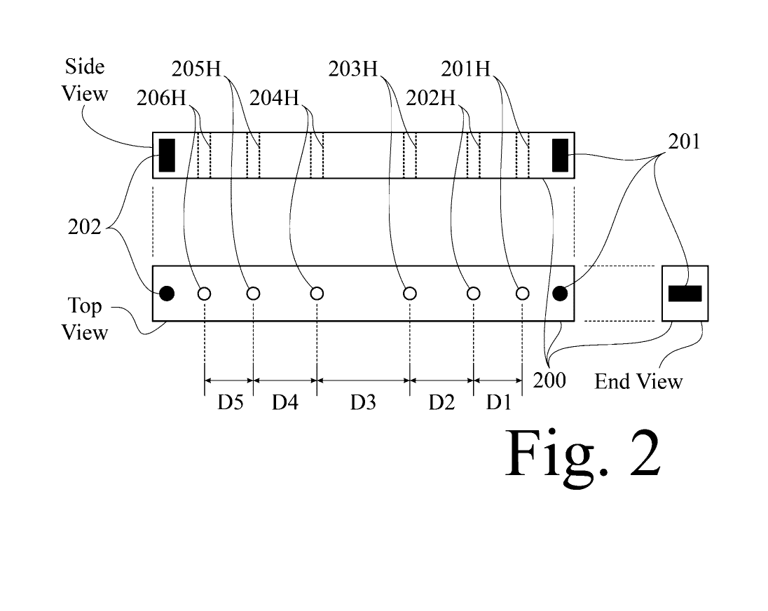

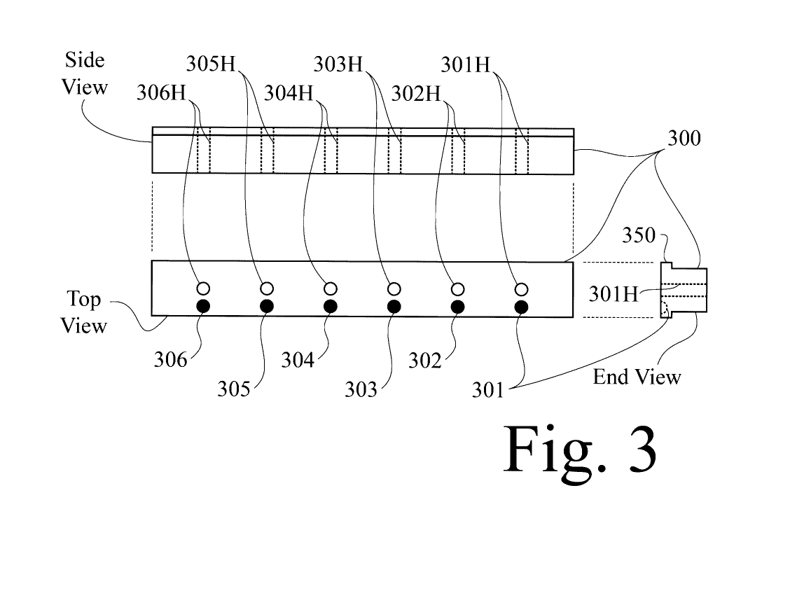

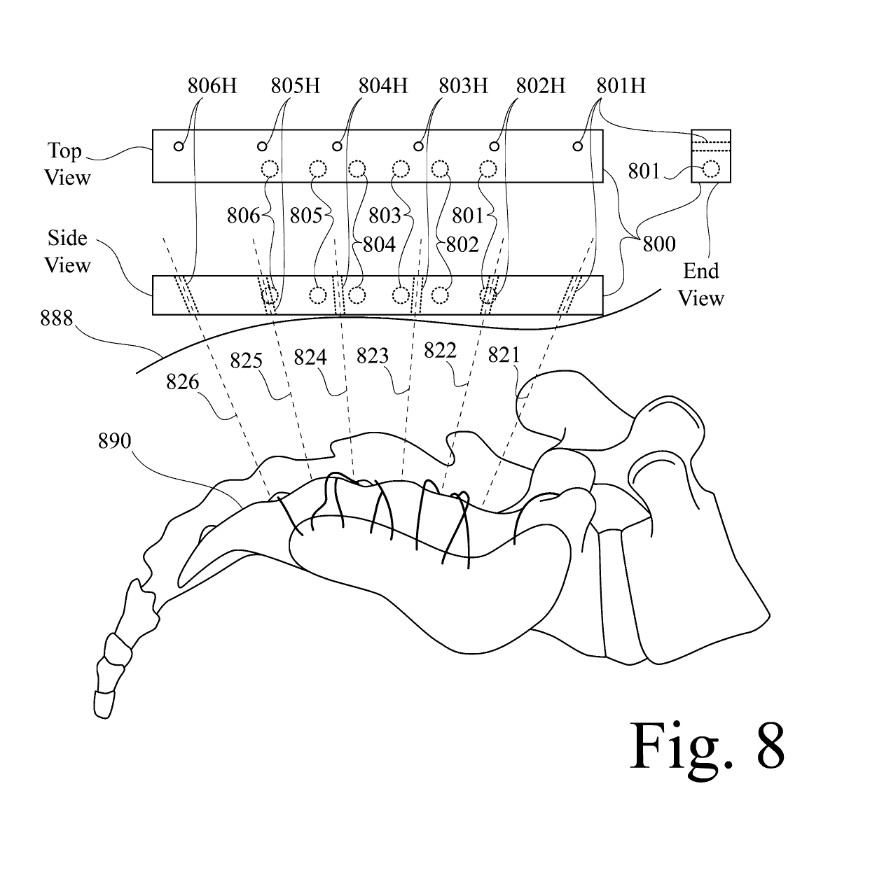

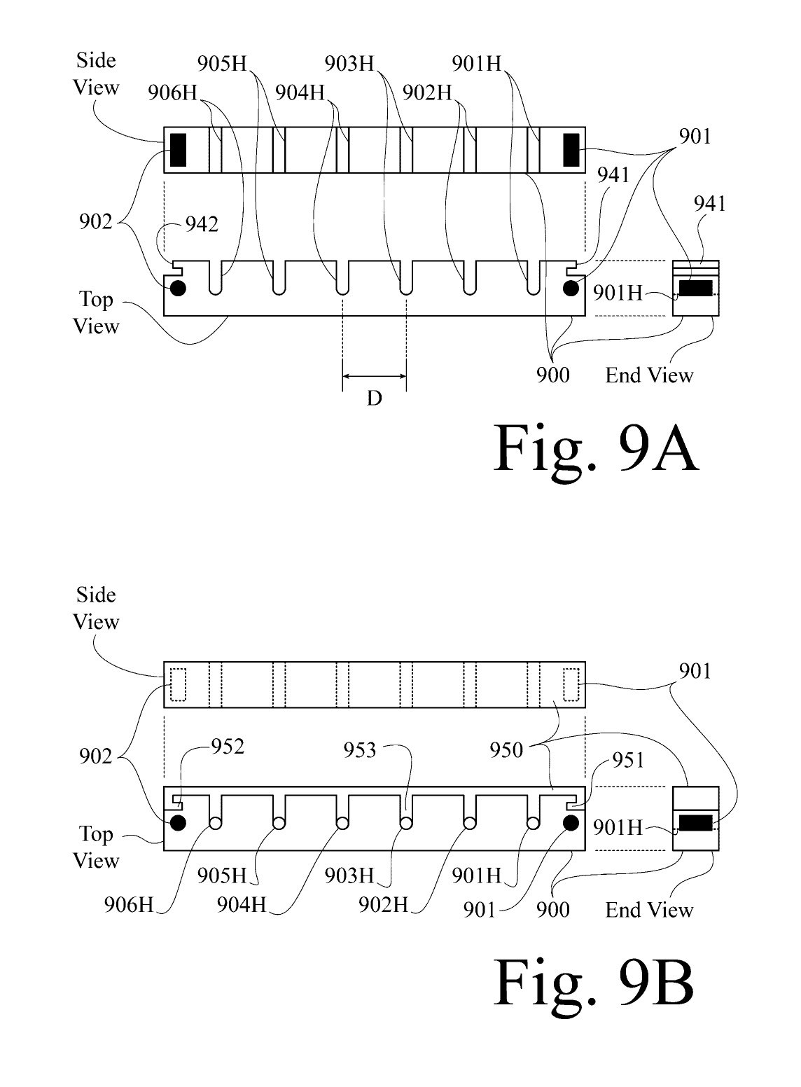

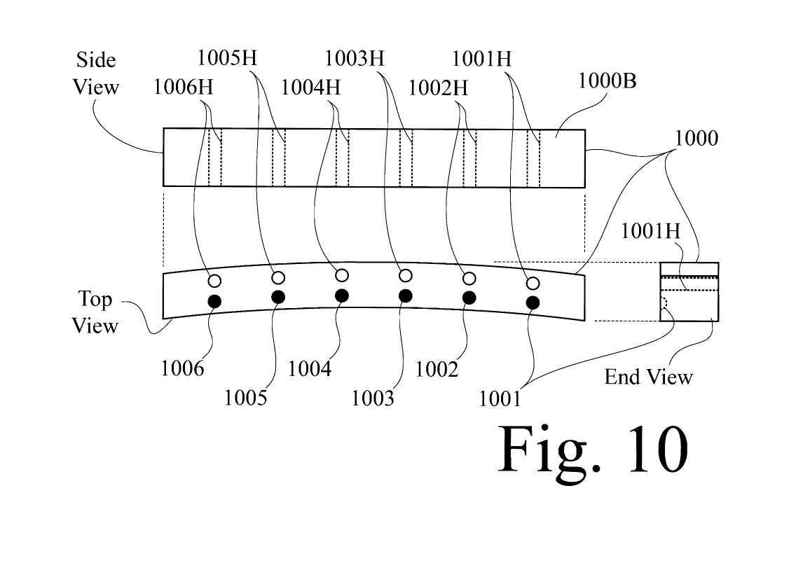

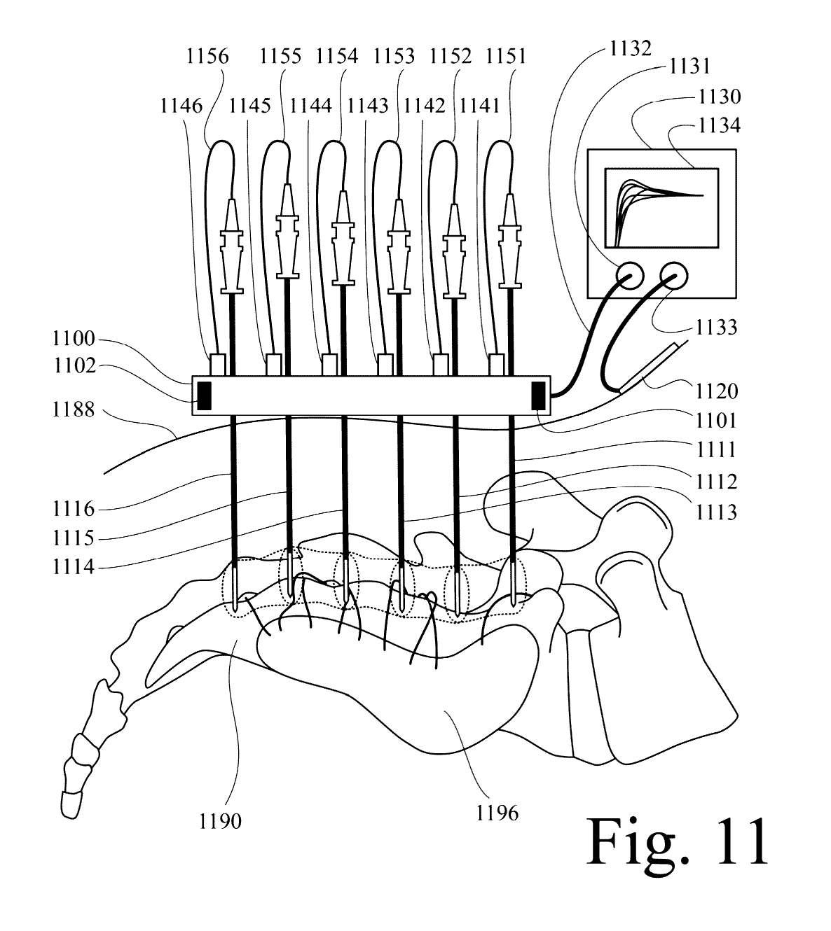

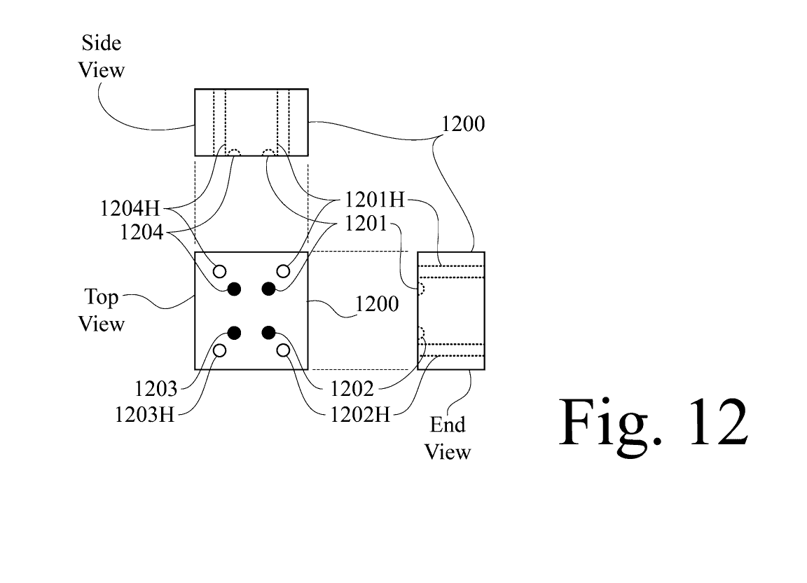

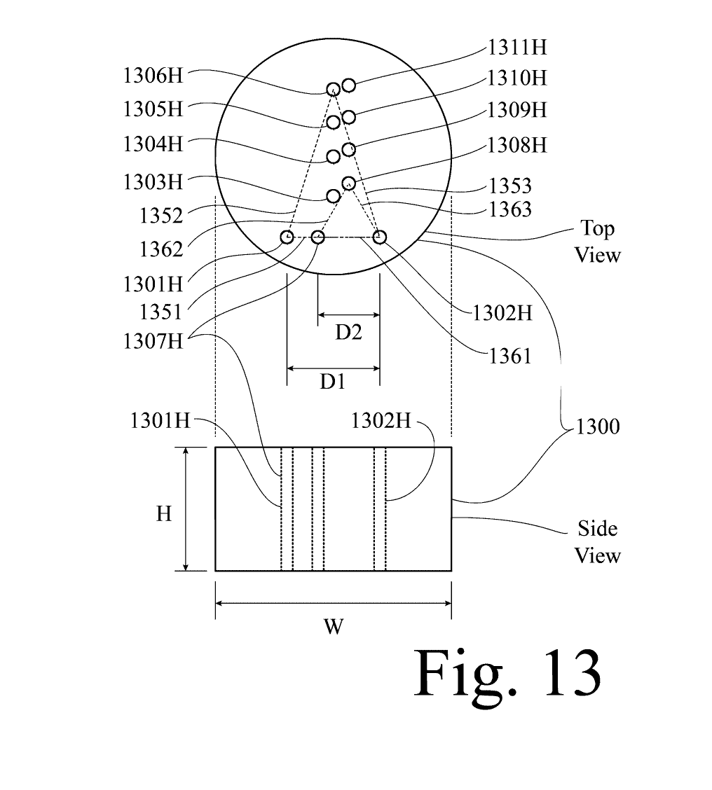

Referring to FIG. 1, in some embodiments, the total number of slots in the guideblock 100 can be a number selected from the group: 2, 3, 4, 5, 6, 7, 8, 9, 10, a number configured to produce a lesion zone covering an anatomical region using generator settings and ablation probe types configured to suit clinical needs, an integer greater than 10. In some embodiments, the guide block can include slots at regular intervals, such as once every 1 mm, one every 2 mm, or one every 5 mm, to provide for the user to adjust the spacing between ablation probes; in some embodiments, the guideblock can include a ruler, numbers, or other indication of the spacing between holes. In some embodiments, ablation probes are positioned in some of the slots and energized, while other slots do not contain any ablation probe. In some embodiments, as in the example of FIG. 9, the slots 101H, 102H, 103H, 104H, 105H, 106H can have a lateral opening such that the position an ablation probe can be individually adjusted to suit clinical needs (such as moving a single ablation probe out of a sacral foramina) and so the guideblock 100 can be removed from the ablation probes which are positioned in the body 170 and which have hub at the their proximal ends that prevents removal of the guideblock 100 by sliding it over proximal end of the ablation probes; in some such embodiments, a the guideblock 100 can include a movable gate (such as gate 950 in FIG. 9B) that clamps and prevents movement of each or all of the ablation probes through the lateral openings unless than gate is opened. In some embodiments, the slots 101H, 102H, 103H, 104H, 105H, 106H can be arranged in a straight line, as shown in FIG. 1. In some embodiments, the slots 101H, 102H, 103H, 104H, 105H, 106H can be arranged in a curved line, as shown in FIG. 10, for example to conform to target anatomy. In some embodiments, the slots 101H, 102H, 103H, 104H, 105H, 106H have unequal spacing, such as shown in FIG. 2. In some embodiments of guideblock 100, an individual x-ray visible maker can be included for each slot 101H, 102H, 103H, 104H, 105H, 106H, such as shown in FIGS. 3, 4, 5, 6, 7, and 8. In some embodiments, the slots 101H, 102H, 103H, 104H, 105H, 106H can produce non-parallel probe arrangements, such as shown in FIG. 8. In various embodiments, the length of the guideblock 100 can be in the range 10-150 mm or longer, depending on the number of ablation probes the guideblock carries, the relative angle of the ablation probes, the inter-probe separation, and other dimensions. The height of the guideblock 100 in the direction of the ablation probes can be in the range 5 mm to 2 cm or taller, and configured to restrain the relative trajectories of the ablation probes (for example to a toleration of +/−1 degree, +/−2 degrees, +/−5 degree, or a tolerance configured to produce reliable inter-tip bipolar heating) for insertion to desired depths (for example in the range 5 cm-20 cm or farther) within the tissue 170. The width of the guideblock 100 in the direct perpendicular to the direction of the ablation probes and perpendicular to the orientation of the line of ablation probes can be in the range 5 mm to 2 cm or wider, and configured to stabilize the ablation probes when they are either partially or fully inserted in the tissue of body 170. In some embodiments, the cross-section (width by height) of the guideblock 100 can have a shape selected from the group: square, rounded rectangle, rectangle, T-shaped, shape configured to provide for support of ablation probes and orientation of ablation probes. In some embodiments, the guideblock 100 can provide connection between the electrosurgical generator and the ablation probes, both for conduction HF energy from the generator to the ablation probes, and for conducting measurement signals from the ablation probes to the generator, such as temperature and impedance signals, such as shown in FIG. 11. In some embodiments, the guideblock 100 can arrange multiple ablation probes in a non-linear arrangement such as a triangle, a square (as shown in FIG. 12 for example), a pentagon, a hexagon, a rectangle, or another shape; this can have the advantage of surrounding a target structure and providing for ablation around and/or across the target structure. In some embodiments, the guideblock 100 can align multiple ablation probes in two or more parallel linear arrangements.

Referring to FIG. 1, in some embodiments, x-ray images can be collected in one or more of the image types selected form the group: anterior-posterior (AP), lateral, posterior-anterior (PA), oblique, and other imaging angles to suit clinical needs as is familiar to one skilled in the art of x-ray imaging. In some embodiments, the x-ray visible markers can be omitted form the guideblock 100. In some embodiments, the guideblock 100 can be configured for use with ultrasound guidance. A two-hole guideblock (with or without x-ray markers) can also be advantageous to align two or more ablation probes for ultrasound-guided ablation, such as monopolar RF ablation or bipolar RF ablation, wherein a geometric relationship between ablation probes is desired so that the shape of heated tissue conforms reliably to the target anatomy, because alignment of more than one ablation probe can be difficult when one of the physician's hands is occupied with holding the ultrasound transducer. In some embodiments, the guideblock 100 can be configured for use with one or more types of image guidance selected form the group: x-ray, fluoroscopy, CT, MRI, PET, ultrasound. In some embodiments, one of the guideblocks 200, 300, 400, 500, 600, 700, 800, 900, 1000, 1100, or 1200 can be used in place of the the guideblock 100 shown in FIG. 1. In some embodiments, the target anatomy can one or more target regions selected from the group: bone, nerve within bone, basivertebral nerve, vertebra, osteoid osteoma, tumor, peripheral nerve, spinal nerve, medial branch nerve, doral ramus of a spinal nerve, dorsal nerve root, dorsal root ganglion, cervical spine, thoracic spine, lumbar spine, sacral spine, hip, joint, knee, shoulder, foot, leg, inguinal region, arm, wrist, carpel tunnel, kidney, lung, liver, pancreas, prostate, uterus, testicle, thyroid, adrenal gland, brain, spinal cord, central nervous system, trigeminal nerve sphenopalatine nerve, sympathetic chaim, ramus communicans, intervertebral disc, nerve within the intervertebral disal, annulus of the intervertebral disc, nucleus of the intervertebral disc, posterior intervertebral disc, sacral lateral branch nerve, L5 dorsal ramus, dorsal innervation of the SIJ, anterior innervation of the SIJ. In some embodiments, the ablation probes can be used with side-on or point-on approaches to a medial branch nerve of the cervical, thoracic, or lumbar regions of the spine. In some embodiments, the system and methods of FIG. 1 can be used in percutaneous, open, and laparoscopic surgical procedures. In some embodiments, the ablation probes 111, 112, 113, 114, 115, 116 can be energized in one or more configurations selected from the group: monopolar wherein an ablation probe is electrically referenced to a dispersive ground pad electrode on the skin surface, monopolar such that all probes are at the same electrical potential at the same time, monopolar sequential such that multiple probes are energized in a monopolar configuration at different times, bipolar, bipolar such that each probe is connected to one of at least two electrical potentials at the same time and current flow between ablation probes, bipolar sequential such that current flows between pairs of electrodes at different times, rapidly repeating sequences of monopolar and/or bipolar configurations, non-repeating sequences of monopolar and/or bipolar configurations wherein heating for one lesion is completed before heating for the next lesion is initiated, and other configurations polarity and switching timing. In some embodiments, the electrosurgical generator controls the temperature at one or more ablation probe. In some embodiments, the electrosurgical generator does not control the temperature at any ablation probe. In some embodiments, one or more heat lesions are generated by each electrode at the same time, and the temperature of each probe is controlled. In some embodiments, pulsed radiofrequency energy is delivered, as is familiar to one skilled in the art of radiofrequency pain management, and the gross tissue temperature is held below the minimum neurolytic range 45-50 degC, for example at a temperature of 42 degC, in either a monpolar or a bipolar configuration. In some embodiments, the slots 101H, 102H, 103H, 104H, 105H, 106H can have a cross-section that is a shape selected from the group: square, rectangular, circular, triangular, hexagonal, or other shape configured to facilitate passage of an ablation probe through the guideblock 100. In some embodiments, a triangular, square, or other polygonal cross section for the slots can have the advantage of proving stability for the ablation probes, and clearance for bodily tissue that adheres to the shaft of a probe and causes difficulty in moving the probe through the slot. In some embodiments, the output signal of the electrosurgical generator can include a low-frequency signal, such a DC signal, configured to heat tissue. In some embodiments, the ablation probes can be connected to a nerve-stimulation signal generator to perform sensory or motor nerve stimulation, either by passing he stimulation signal between ablation probes in a bipolar manner, or between an ablation probe and a reference ground pad in a monopolar configuration.

Referring now to FIG. 2, in accordance with several aspects of the present invention, a guideblock 200 includes radiopaque markers 201, 202 and parallel slots 201H, 202H, 203H, 204H, 205H, 206H with unequal inter-slot spacings D1, D2, D3, D4, D5, for parallel alignment of multiple ablation probes within a living body. Spacing D1 is the distance between slots 201H and 202H. Spacing D2 is the distance between slots 202H and 203H. Spacing D3 is the distance between slots 203H and 204H. Spacing D4 is the distance between slots 204H and 205H. Spacing D5 is the distance between slots 206H and 206H. In the embodiments shown in FIG. 2, the spacing between electrode slots increases toward the end of the row of slots. In particular, the distances statisfy the relationship D3>D2>D1 and D3>D4>D5. This has the advantage that when an ablation probe is placed in a body through each slot, and all ablation probes heat tissue at the same time, the heating distribution along the row of ablation probes is uniform because heat tends to be more condensed by thermal diffusion toward the middle of the row, and heat tends to be more dispersed by thermal diffusion toward the ends of the row. For example, in the case of heating between each adjacent pair in a bipolar configuration using RF current, larger inter-electrode spacing can produce sufficient inter-electrode tissue heating the closer a pair of bipolar electrodes is to the middle of the row of RF electrodes. In the example shown in FIG. 2, the following relationships apply: D1=D5 and D2=D4. In other embodiments, the inter-slot spacings can be arranged in a different pattern to suit clinical objectives for a particular target anatomy and electrode type. In some applications using a straight-line row of guide-holes 201H, 202H, 203H, 204H, 205H, 206H, only two radiopaque markers are sufficient to indicate the orientation and extent of the guideblock relative to anatomy in x-ray imaging. In some embodiments of guideblock 200, the spacings can be D3=15 mm, D2=D4=12 mm, D1=D5=10 mm.

Referring now to FIG. 3, in accordance with several aspects of the present invention, a guideblock 300 includes radiopaque markers 301, 302, 303, 304, 305, 306 positioned proximate to parallel slots 301H, 302H, 303H, 304H, 305H, 306H, respectively, each of which is provides for alignment of an ablation probe and/or needle into the living body in a straight row. Each marker produces can x-ray image that indicates the location one of the guide-holes relative to anatomy, eg marker 301 indicates the position of guide-hole 301H. A physician can use guideblock 300 to plan the placement of each of up to six ablation probes, such as RF cannulae, in a patient's body. One method of placement of two or more ablation probes in a geometric arrangement within the living body includes imaging a guideblock in relation to a living body, determining the target location of each ablation probe by the image of a radiopaque marker registered to each slot in the guideblock, and inserting each ablation probe through a slot in the guideblock. Dotted lines of the slots 301H, 302H, 303H, 304H, 305H, 306H in the Side View, and of the slot 301H and marker 301 in the End View show aspects of these features that are internal to the guideblock relative to the displayed outer surface. The block 300 also includes ergonomic feature 350 to facilitate manipulation of the block 300 by hand.

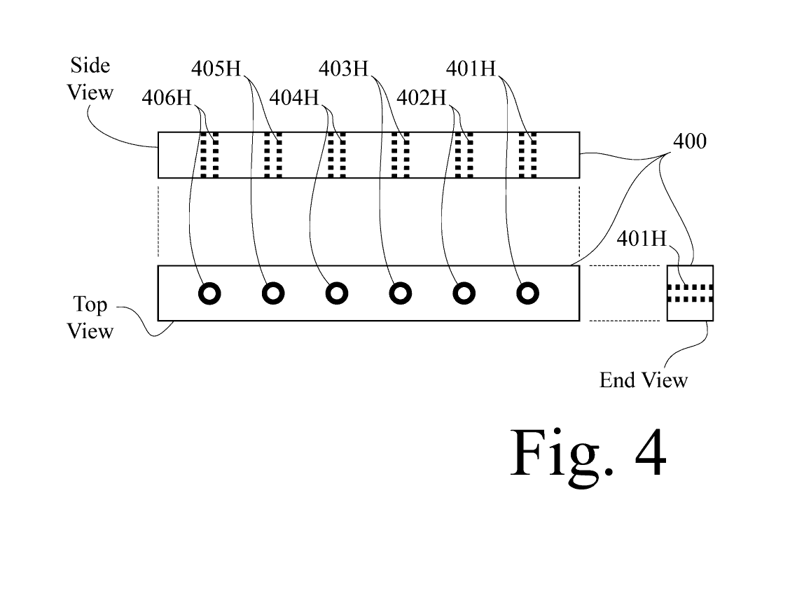

Referring now to FIG. 4, in accordance with several aspects of the present invention, a guideblock 400 includes parallel slots 401H, 402H, 403H, 404H, 405H, 406H, which each comprise an x-ray-visible structure. In some embodiments, each slot is an x-ray visible tube, for example a metal tube or a tube formed from a plastic material that is impregnated with metal such as tungsten. One advantage of the embodiment of a guideblock 400 presented in FIG. 4 is that the x-ray marker and guidehole are an integral piece to provide for accurate indication of the ultimate location of a needle passing through each guidehole 401H, 402H, 403H, 404H, 405H, 406H.

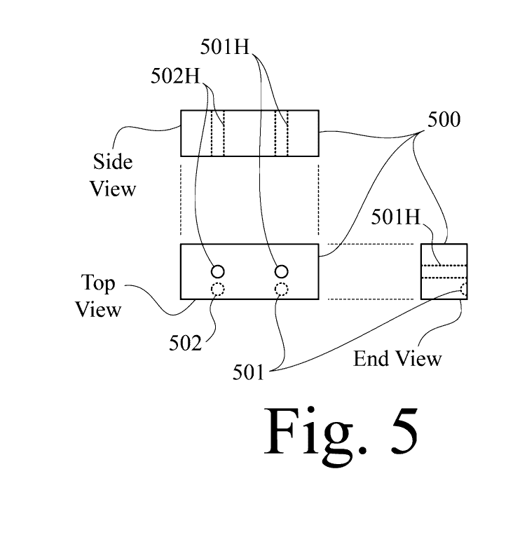

Referring now to FIG. 5, in accordance with several aspects of the present invention, a guideblock 500 includes radiopaque markers 501, 502 and parallel slots 501H, 502H, for parallel alignment of two ablation probes within a living body. One advantage of a guideblock with only two holes is that it can simplify bipolar RF ablation between two RF ablation probes, such as ablation of the thoracic medial branch nerve at the T5-T8 levels where the medial branch nerve is located in the inter-transverse-process tissue without regular reference to bony landmarks, or for a lateral approach to bipolar ablation of the cervical medial branch. A two-hole guideblock (with or without x-ray markers) can also be advantageous to align two ablation probes for ultrasound-guided bipolar RF ablation, which can be difficult because one of the physician's hands is occupied with holding the ultrasound transducer. One advantage of this embodiment of the a guideblock is that an extended lesions zone without gaps can be generating using only two ablation probes by first generating one or more lesion in a first position using two ablation probes inserted through the guideblock 500, removing the first ablation probe, rotating the guideblock 500 to a new location with the second ablation probe as a reference, reinserting the first ablation probe through the guideblock 500 into a new location, generating one or more lesions in the said new location. This process can be repeated one or more times by removing either the first ablation probe or the second ablation probe, and rotating the guideblock 500 to a new location using the other ablation probe as a reference. In one example, at each location, either a monopolar or a bipolar lesion can be created. This general process can be used to create a wide variety of lesion zones, including an elongated row of lesions (by “leap-frogging” ablation probes along a straight path), a curved row of lesions (by “leap-frogging” ablation probes along a curved path), a circular series of lesions (by “leap-frogging” ablation probes along a curved path that ends where it began), a cylindrical lesion zone (by repeatedly rotating the guideblock 500 around the same ablation probe). In one important example, the first and second ablation probes are RF electrodes that are energized in a bipolar manner at each location. One important advantage of this method is that the sequential lesions can be guaranteed to overlap because each lesion can share the location of one of the ablation probes. The inclusion of radiopaque markers 501, 502 has the advantage that a physican can use x-ray to plan the multi-step process of generating multiple overlapping lesions to conform to patient anatomy. In some embodiments, guideblock 500 can further include additional slots and matching markers, and the process can be performed wherein all but one ablation probe are removed so that the guideblock can be rotated into a new position between lesions.

One method (hereinafter “Method A”) of generating heat lesions in a living body using two or more ablation probes comprises putting a guideblock including at least two slots in a first position relative to a body, inserting a first ablation probe through a first slot into a first location in the body, inserting a second ablation probe through a second slot into a second location in the body, generating one or more heat lesions in the body by means of one or more ablation probes, withdrawing either the first ablation probe or the second ablation probe from the body, rotating the guideblock around the ablation probe that was not withdrawn to a second position relative to the body, reinserting withdrawn ablation probe into a third location in the body through the slot from which it was withdrawn, generating one or more heat lesions in the body by means of one or more ablation probes. One method (hereinafter “Method B”) comprises Method A and further comprises repeatedly withdrawing either the first ablation probe or the second ablation probe from the body, rotating the guideblock 500 around the ablation probe that was not withdrawn to a new position relative to the body, reinserting withdrawn ablation probe into the body through the slot from which it was withdrawn, generating one or more heat lesions in the body by means of one or more ablation probes. In some embodiments of Method A, putting guideblock in a position relative to a body includes generating an x-ray image that of the guideblock in relation to the body, and identifying one or both of the x-ray markers. In some embodiments of Method A, the ablation probes are RF ablation probes and the heat lesions include bipolar RF heat lesions. In some embodiments of Method A, the heat lesions include the sacral lateral branch nerves. Some embodiments of Method A are methods of treating SIJ pain. In some embodiments wherein the ablation probes are RF ablation probes and the heat lesions include bipolar RF heat lesions. In some embodiments of Method A, withdrawing either the first ablation probe or the second ablation probe from the body includes withdrawing all ablation. Some embodiments of Method A further comprise: when the guideblock is in the first position, inserting one or more additional ablation probes through one or more additional slots in the guideblock, respectively, into the body; withdrawing all additional ablation probes from the body when withdrawing either the first ablation probe or the second ablation probe from the body; and reinserting zero or more of the additional ablation probes through the additional slots in the guideblock when reinserting said withdrawn ablation probe.

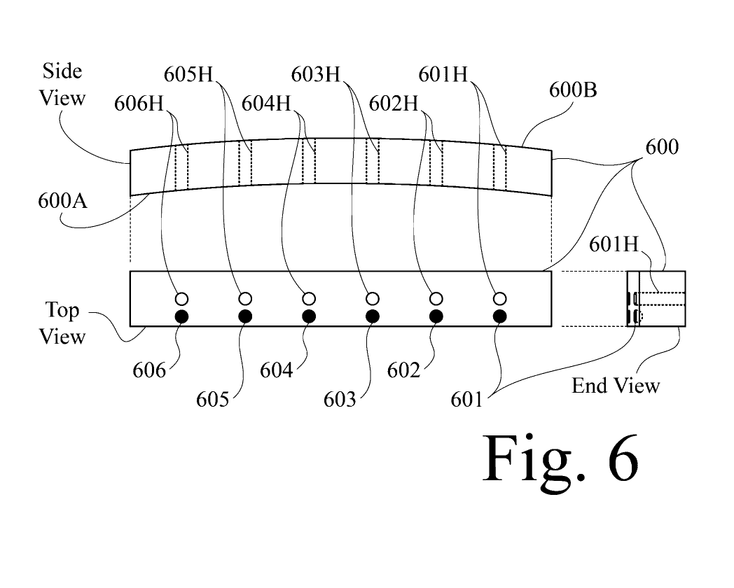

Referring now to FIG. 6, in accordance with several aspects of the present invention, a guideblock 600 includes a curved surface 600A and radiopaque markers 601, 602, 603, 604, 605, 606 positioned proximate to parallel slots 601H, 602H, 603H, 604H, 605H, 606H, respectively, each of which is provides for alignment of an ablation probe and/or needle into the living body in a straight row. In some embodiments, the curved surface 600A can be the surface that touches the body into which ablation probes are inserted through the slots 601H, 602H, 603H, 604H, 605H, 606H, and the surface 600A can be configured to conform to shape of the living body where the guideblock touches the living body. For example, the surface 600A can be curved to conform to the curvature of the skin over the sacroiliac joint region in a human patient body. One advantage of a guideblock that includes a conformal tissue-interface surface in that positioning of the guideblock relative to target anatomy of the body is facilitated.

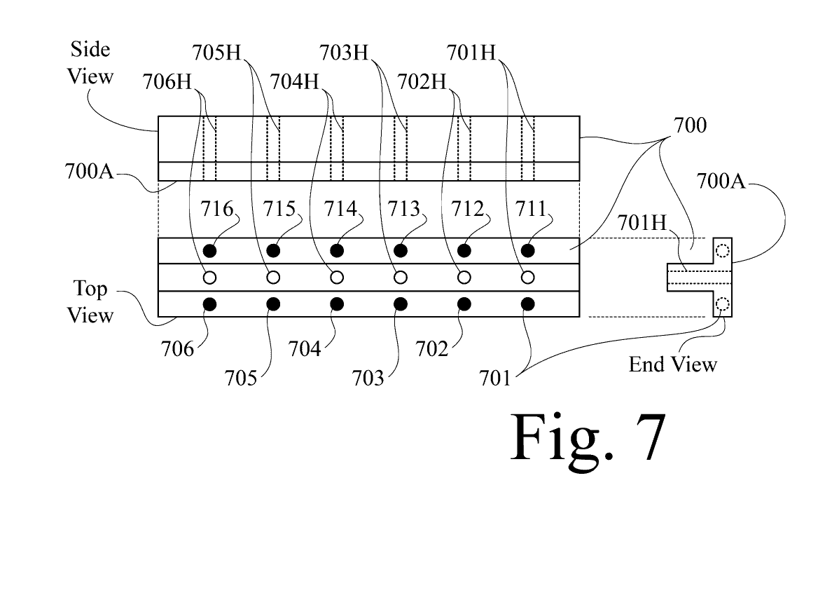

Referring now to FIG. 7, in accordance with several aspects of the present invention, a guideblock 700 includes radiopaque markers 701, 702, 703, 704, 705, 706, 711, 712, 713, 714, 715, 716 positioned proximate to parallel slots 701H, 702H, 703H, 704H, 705H, 706H, each of which is provides for alignment of an ablation probe and/or needle into the living body in a straight row. Each slot 701H, 702H, 703H, 704H, 705H, 706H has two markers aligned with it, the two markers being on opposite sides of their corresponding slot. For example, slots 706 and 716 correspond to slot 706H. Guideblock further includes a T-shaped cross section. The T-shaped cross section can provide for easy distinction of the top and bottom of the guideblock 600; increased guideblock and slot height for increased guidance and support of each ablation probe inserted through a slot without a large increase in guideblock weight; and increased guideblock width for increased stability, without a large increase in guideblock weight for embodiments of the guideblock 700 wherein the surface 700A is the bottom surface of the guideblock positioned on top of a body surface.

Referring now to FIG. 8, in accordance with several aspects of the present invention, a guideblock 800 includes radiopaque markers 801, 802, 803, 804, 805, 806 positioned to indicate the distal tip locations of ablation probes inserted to a particular depth in a body through slots 801H, 802H, 803H, 804H, 805H, 806H, respectively, each of which is provides for alignment of an ablation probe and/or needle into the living body in a straight row wherein ablation probes are not all parallel to one another. Paths 821, 822, 823, 824, 825, 826 indicate the non-parallel trajectories that are set by through-holes 801H, 802H, 803H, 804H, 805H, 806H, respectively, which are configured to guide multiple ablation probes or needles to be substantially perpendicular to the dorsal surface of the sacrum 890, which is one example of an internal bodily target structure. The typical curvature of the human sacrum 890 and distance between the dorsal sacrum 890 and skin surface 888 posterior influence the spacing and orientation of the slots 801H, 802H, 803H, 804H, 805H, 806H. The markers 801, 802, 803, 804, 805, 806 are configured to indicate the intersection of the needle paths 821, 822, 823, 824, 825, 826, respectively, with the dorsal sacral surface 890 when viewed by means of an AP or PA x-ray image. One advantage of a guideblock that includes non-parallel holes is that ablation probes can be aligned with internal structures that have irregular and/or curved geometry; one example of this is the dorsal surface of the sacrum where creation of a series of bipolar lesions to interrupt the sacral lateral branch nerves to treat SIJ pain by placing RF cannula electrodes more perpendicular to the curved sacral surface can minimize inter-tip offsets as described in Cosman and Gonzalez (2011). One advantage of a guideblock that includes non-parallel holes is that ablation probes and/or needles can avoid structures along their path to target positions in the body. For example, it can be clinically useful to place the active tips of multiple RF ablation probes nearby each other to create an enlarged cluster lesion zone by heating tissue with the multiple RF ablation probes at the same time (for example, by bringing all active tips to the same electrical potential in a “cluster” configuration, or by driving current between the active tips in one or more “bipolar” configurations); however, the presence sensitive anatomical structures (such as a large blood vessel or sensitive organ) or hard anatomical structures (such as bony structures like the ribs) that are more superficial to the target location of the active tips can hinder parallel placement of the ablation probes. One example of this situation is creating large cluster RF lesions in and around tumors in large organs, like the liver, of which some parts are surrounded by the ribs that are not easily penetrated by the ablation probes.

FIG. 9 refers collectively to FIGS. 9A and 9B. Referring now to FIG. 9, in accordance with several aspects of the present invention, a guideblock 300 includes radiopaque markers 901, 902 positioned at the ends of a row of parallel slots 901H, 902H, 903H, 904H, 905H, 906H, respectively, each of which is provides for alignment of an ablation probe and/or needle into the living body in a row, and each of which include a lateral opening which allows for removal of the guideblock 900 after ablation probes have been positioned in a body through the guideblock 900, and for lateral adjustment of each ablation probe position to suit clinical needs. In this example, the inter-slot spacing D is equal. The guideblock 900 additionally includes rails 941 and 942 that mate with complementary rails 951 and 952, respectively, of clamping plate 950, which closes off the lateral opening of each slot 901H, 902H, 903H, 904H, 905H, 906H and thereby constrains the position of ablation probes inserted through the slots 901H, 902H, 903H, 904H, 905H, 906H. In some embodiments, the clamping plate 950 can provide for tight clamping each ablation probes. In some embodiments, the clamping plate 950 can provide for loose clamping each ablation probes. The clamping plate can be mated to the block 900 during placement of ablation probes into a body to constrain the probe trajectories, and then once the probes are self-supported by the bodily tissue, the plate can be removed laterally.

Referring now to FIG. 10, in accordance with several aspects of the present invention, a guideblock 300 includes radiopaque markers 1001, 1002, 1003, 1004, 1005, 1006 positioned proximate to parallel slots 1001H, 1002H, 1003H, 1004H, 1005H, 1006H, respectively, each of which is provides for alignment of an ablation probe and/or needle into the living body in a row that is not straight (for example, curved) to conform to non-straight target anatomy.

Referring now to FIG. 11, in accordance with several aspects of the present invention, a guideblock 1000 includes radiopaque markers 1101, 1102 indicating the geometry of parallel slots each of which is provides for alignment of an ablation probe and/or needle in the living body to produce a straight row of parallel ablation probes 1151, 1152, 1153, 1154, 1155, 1156. Each ablation probe 1151, 1152, 1153, 1154, 1155, 1156 passes through the skin 1188 and the ablation probe active tips are at or near the dorsal surface of the sacrum 1190. The guideblock 1100 further include a connection jack 1141, 1142, 1143, 1144, 1145, 1146 for each ablation probe 1151, 1152, 1153, 1154, 1155, 1156, respectively, and a connection cable 1132 to the multi-output jack 1131 of multi-electrode HF generator 1130; and the guideblock 1100 conducts HF electrical signal output from the HF generator to each of the ablation probes 1151, 1152, 1153, 1154, 1155, 1156. In different embodiments and/or modes of operation, the guideblock and the generator can apply a variety of electrical potential configurations to the ablation probes, including monopolar configurations (wherein an ablation probe is reference to a ground pad 1120 placed on the skin surface 1188), bipolar configurations (wherein current passes between ablation probes), and combinations thereof, including sequential and simultaneous combinations thereof configured to generate a desired geometry for the total lesioned tissue. The cable 1132 can be inseparably connected to the block 1100, or separable connected to the block 1100. In the embodiment shown in FIG. 11, generator 1130 is an RF generator (that can further include nerve stimulation output, as is known in the part of pain management RF ablation) and a bipolar heat lesions is generated between each adjacent pair of ablation electrodes 1151, 1152, 1153, 1154, 1155, 1156, each of which includes a temperature sensor whose signal is monitored, controlled, and displayed on screen 1134. In some embodiments, all said bipolar heat lesions can be generated at the same time. In some embodiments, all said bipolar heat lesions can be generated sequentially. In this example, the ablation probes placement and lesion geometry is configured to interrupt the dorsal innervation of the SIJ 1196; in other examples, other anatomical targets and clinical objectives can achieved by the present invention. In FIG. 11 the ablation probes 1151, 1152, 1153, 1154, 1155, 1156 have electrically-insulated shafts, are physically separate from the guideblock 1100, and are connected to the guideblock electrically via cables connected to jacks 1141, 1142, 1143, 1144, 1145, 1146, respectively. In some embodiments, the ablation probes 1151, 1152, 1153, 1154, 1155, 1156 are inseparable, slideably mated with the guideblock 1100; this has the advantage of being a single, integral tool for SIJ ablation. In some embodiments, each ablation probes 1151, 1152, 1153, 1154, 1155, 1156 are have a proximal portion of its shaft that is uninsulated and electrically coupled to a contact in the guideblock slot through which it is passing, the contact providing HF output from the HF generator 1130; in this embodiment a collapsible shroud can cover electrically-uninsulated area of the probe shaft.

Referring now to FIG. 12, in accordance with several aspects of the present invention, a guideblock 1200 includes radiopaque markers 1201, 1202, 1203, 1204 positioned proximate to parallel slots 1201H, 1202H, 1203H, 1204H, respectively, each of which is provides for alignment of an ablation probe and/or needle into the living body. The guideblock 1200 can align four ablation probes in a rectangular array; this array shape can also be considered as two straight, parallel rows. The guideblock 1200 can align three ablation probes in a right-triangular array. The guideblock 1200 can align two ablation probes in a parallel. Each marker produces can x-ray image that indicates the location one of the guide-holes relative to anatomy, eg marker 1201 indicates the position of guide-hole 1201H. A physician can use guideblock 1200 to plan the placement of each of up to four ablation probes, such as RF cannulae, in a patient's body. In some embodiments, the number of guideholes can be 2, 3, 4, 5, 6, 7, 8, 9, 10, or more. In some embodiments, the array can have a geometric shape selected from the group: triangle, square, rectangle, two parallel rows, two straight parallel rows, two curved parallel rows, two or more parallel rows, and other shapes. One advantage of the guideblock 1200 is that ablation probes can be positioned around a target structure. One advantage of the guideblock 1200 that can array probes in two parallel rows is that a thicker row of lesions can be generated, for example to reduce blood supply for bloodless tissue resection in an organ such as the liver or kidney, or to interrupt a longer length of nerves that pass roughly perpendicular to the row (eg sacral lateral branch nerves) is that ablation probes can be positioned.

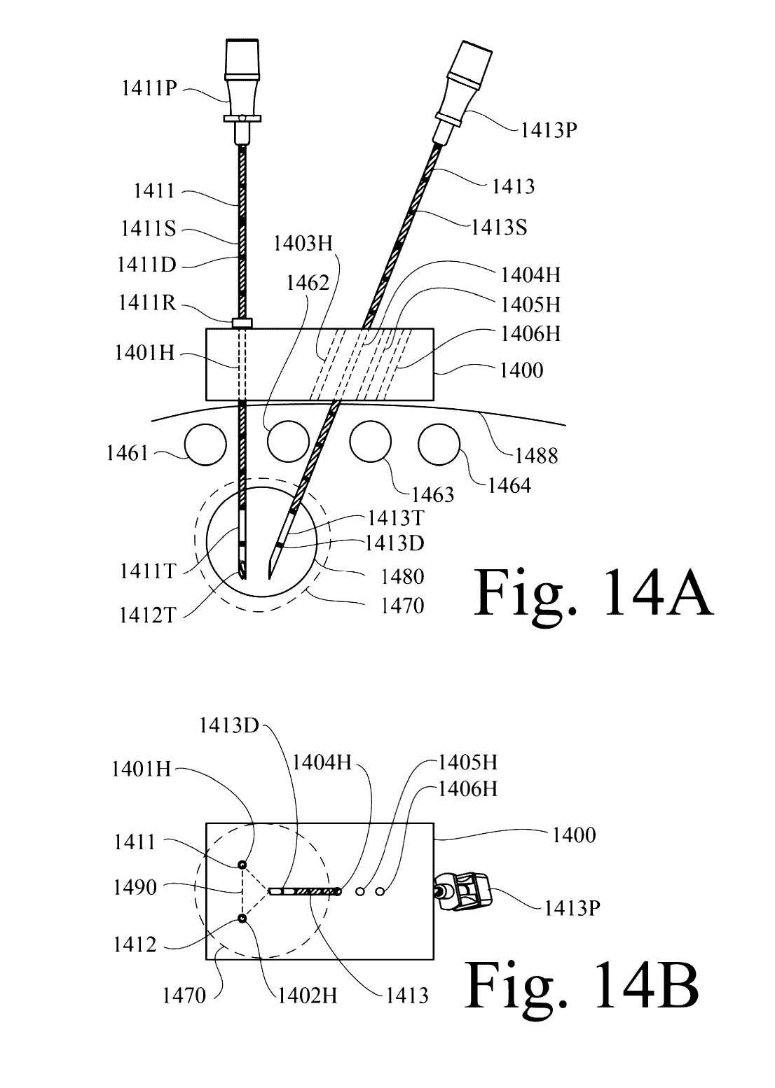

Referring now to FIG. 13, in accordance with several aspects of the present invention, a cylindrical guideblock 1300 with radius W includes parallel slots 1301H-1311H, each of which is provides for alignment of an ablation probe and/or needle in the living body in a parallel-probe array. The guideblock can align three ablation probes in a variety of triangular arrays, including equilateral and isosceles triangles with various spacing. Holes 1301H and 1302H can set a first base distance D1 for a variety of isosceles triangle arrays with one of holes 1303H, 1304H, 1305H, 1306H. For example, the dashed-line triangle with sides 1351, 1352, and 1353 present one example of a isosceles triangle array that can be effected by placing an ablation probe through each of the slots 1301H, 1302H, and 1306H, wherein the inter-probe distances 1352 and 1353 are equal and larger than the distance 1351 (equal to D1). The slots 1301H, 1302H, and 1304H can be used to create an equilateral triangle probe array. The slots 1301H, 1302H, 1303H can create an isosceles triangle array wherein each of the two sides with the same length is shorter than the third side. The block 1300 also provides for a second base distance D2 between slots 1307H and 1302H, and when ablation probes are placed in each of those holes and either hole 1308H, 1309H, 1310H, or 1311H, an isosceles triangle probe array is produced. For example, placement of three probes in holes 1307H, 1302H, and 1308H produces an equilateral triangle array as indicated by dashed lines 1361, 1362, 1363, each with length D2. In some embodiments, block 1300 can include x-ray-visible markers. In some embodiments, block 1300 can omit x-ray-visible markers. In some embodiments, block 1300 can include markers visible in one or more of the imaging modalities in the group: MRI, CT, x-ray, ultrasound.

In one embodiments of guideblock 1300, slots 1301H-1311H each accommodate a 15 gauge cannula electrode, the block height is 2 cm, D1 is 15 mm, and D2 is 10 mm, and these dimensions are configured for creating large cooled RF ablation regions using three cooled RF electrode energized in a monopolar cluster configuration, wherein each electrode is at the same electrical potential simultaneously. Holes 1301H and 1302H create a triangular array with sides 15 mm-1 0 mm-10 mm using hole 1303H, with sides 15 mm-15 mm-15 mm using hole 1304H, with sides 15 mm-20 mm-20 mm using hole 1305H, and with sides 15 mm-25 mm-25 mm using hole 1306H. Holes 1307H and 1302H create a triangular array with sides 10 mm-10 mm-10 mm using hole 1308H, with sides 10 mm-15 mm-15 mm using hole 1309H, with sides 10 mm-20 mm-20 mm using hole 1310H, and with sides 10 mm-25 mm-25 mm using hole 1311H. In other embodiments, guideblock 1300 can be sized for other cannula electrode sizes, inter-electrode spacings, block thicknesses, and clinical applications for example in the ranges described herein in relation to FIG. 1.

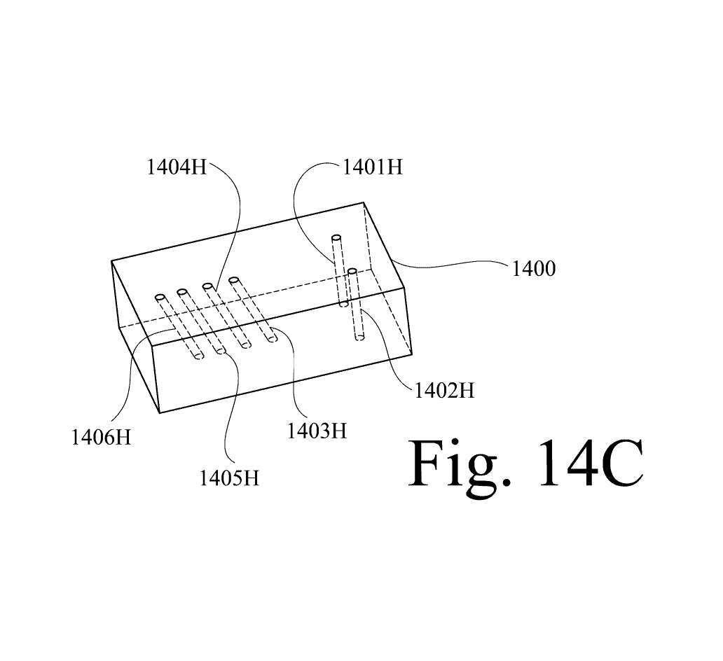

FIG. 14 refers collectively to FIG. 14A, FIG. 14B, and FIG. 14C. FIG. 14A shows a lateral view of a guideblock 1400 aligning three RF cannulae 1411, 1412, 1413 with stylets in place penetrating skin surface 1488 of a body; passing between ribs 1416, 1462, 1463; and penetrating target 1480 within the body. FIG. 14B shows a bottom view of the guideblock 1400 and the RF cannulae 1411, 1412, 1413. FIG. 14C shows a three dimensional view of the guideblock 1400. Referring to FIG. 14, in accordance with several aspects of the present invention, a guideblock 1400 includes slots 1401H, 1402H, 1403H, 1404H, 1405H, 1406H, each of which is provides for alignment of an ablation probe and/or needle in the living body in an array including one or more probes that are not parallel. The guideblock 1400 can align three ablation probes in a variety of triangular arrays, wherein two probes are parallel and one probe is not parallel to the other probes. In one example, guideblock 1400 can be used to place three probes 1411, 1412, 1413 in a cluster configuration in an anatomical target 1480 that are obstructed by the ribs 1461, 1462, 1463, 1464, by passing two parallel probes 1411, 1412 through a first intercostal space, and a third probe 1413 through a second intercostal space using an orientation that is not parallel to orientation of the said two parallel probes 1411, 1412. The target structure 1480 can be a tumor in an organ such as the kidney or lung or another organ with aspects near ribs or other bony or sensitive structures that restrict free orientation of multiple ablation probes. In the example shown in FIG. 14, dashed outline 1470 shows the ablation region that form if the same RF potential is applied to the active tips 1411T, 1412T, 1413T of the cannula 1411, 1412, 1413 by means of internally-cooled RF electrodes placed within the cannulae inner lumen. In some embodiments, conventional, non-cooled RF can be used with the guideblock 1400. In some embodiments, ablation probes 1411, 1412, 1413 can be energized with MW or RF energy or a stimulation signal, in both monopolar, bipolar, and multipolear configurations. The edges of dashed-line triangle 1490 illustrate the geometric relationship between the distal points of the cannulae 1411, 1412, and 1413. Each cannula has an sharpened electrically-conductive active tip at the distal end of the shaft, an electrically-insulated proximal shaft, depth markers along the shaft (indicating 1 cm intervals, in the example shown), a depth stop which the physician can slide along the shaft, and a hub for injection and mating with an ablation electrode; for cannula 1411, these elements are active tip 1411T, hatched region insulation 1411S, depth markers 1411D, depth stop 1411R, and hub 1411P, respectively. In some embodiments, a depth stop can be a ring slideably engaged with an ablation probe shaft. In some embodiments, a depth stop can be a clip that clips to an ablation probe shaft. The use of a guideblock with ablation probes having depth markers, eg 1411D, along their shaft can be advantageous for non-parallel guidehole configurations because the depth markers can indicate that a desired geometric configuration has been achieved even when the distal end of the ablation probes are inserted into and hidden by tissue. A system that includes a guideblock and ablation probes with depth markers is advantageous for planning and achieving desired ablation probe configurations in a body. The said system and further including depth stops on the ablation probe shafts has the additional advantage of ease of use and error free use of depth markers to achieve a multi-electrode geometric arrangement in a body for tissue ablation. In some embodiments, the depth stops can be omitted.

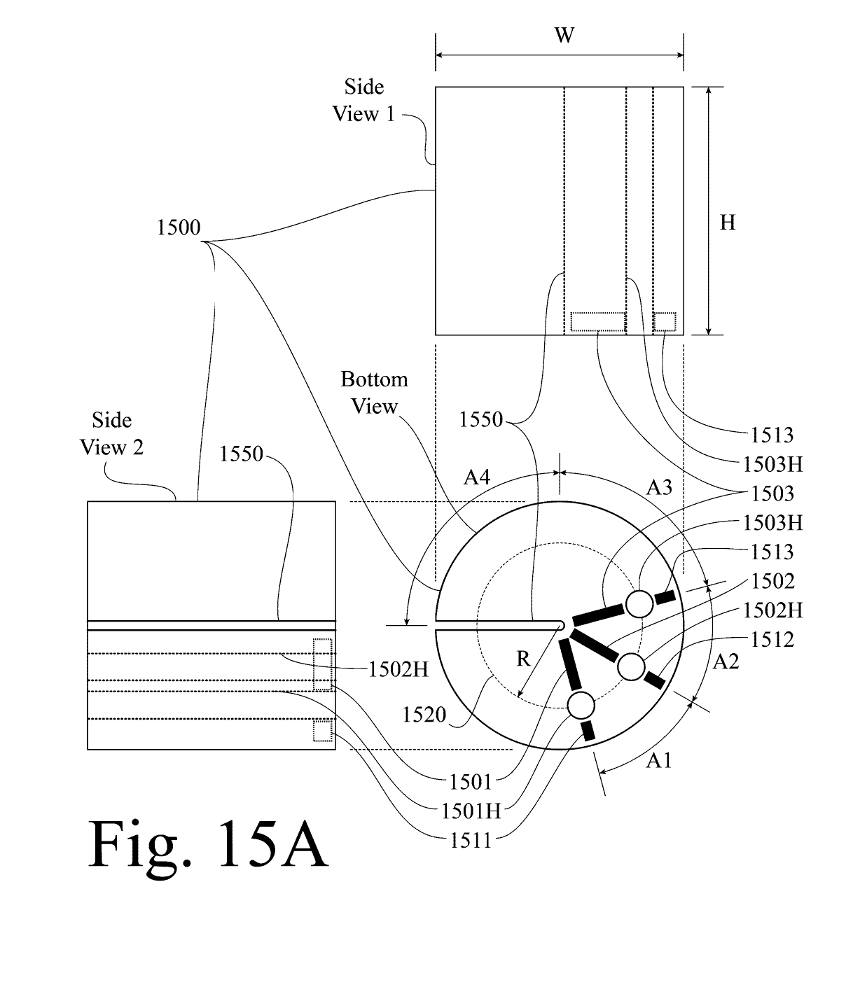

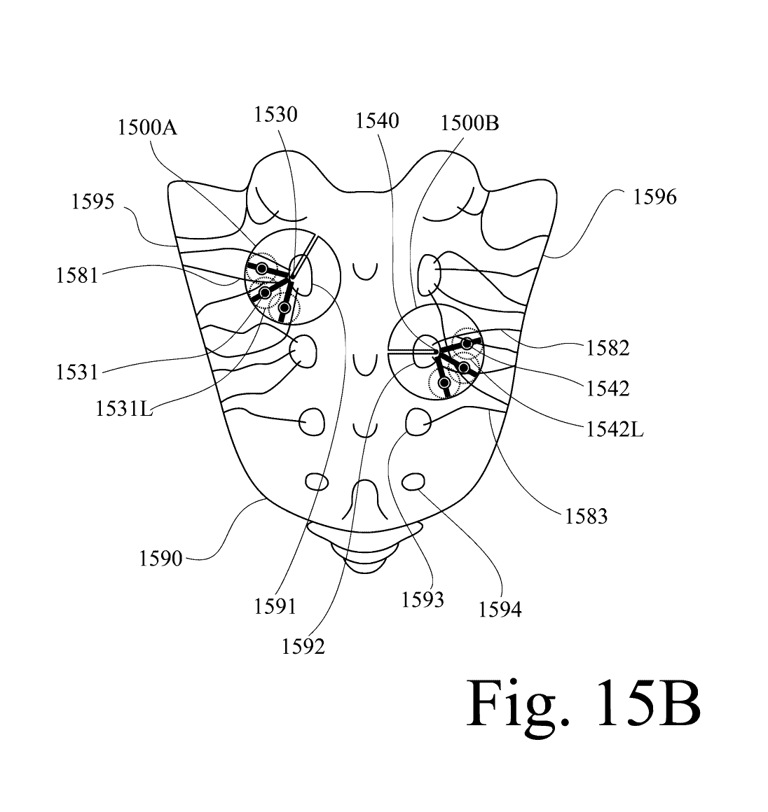

Referring now to FIG. 15A, guideblock 1500 is shown in three perpendicular views (bottom, side 1, side 2 including three parallel ablation-probe slots 1501H, 1502H, 1503H for aligning up to three ablation probes in a body, a guide-needle slot 1550 with lateral opening for aligning a guide needle parallel to the ablation probes, and radiopaque markers 1501, 1511, 1502, 1512, 1503, 1513 for identification of anatomical target location of the ablation probes relative to the guide-needle target. Each slot 1501H, 1502H, 1503H is a clear hole through the guideblock 1500, in the longitudinal direction of cylindrical guideblock 1500, with diameter W and height H. The slots 1501H, 1502H, 1503H are a distance R from the centeral axis of the guideblock, as illustrated by dashed circle 1520. Markers 1501, 1511, 1502, 1512, 1503, 1513 are positioned on the bottom of the guideblock to minimize spread in an x-ray image to due the divergence of the x-ray beam. Slots 1501H and 1502H are spaced by angle A1 relative to the center of circle 1520. Slots 1502H and 1503H are spaced by angle A2 relative to the center of circle 1520. Slots 1502H and 1550 are spaced by angle (A3+A4) relative to the center of circle 1520. The guideblock 1500 can be used in method of tissue ablation comprising placing a guide needle into an anatomical structure, aligning guideblock 1500 relative to the guide needle, aligning one or more ablation probes relative to the guide needle by means of the guideblock, and generating one or more ablations by means of the ablation probes. Markers 1501 and 1511 indicate the location of slot 1501H by the space between the markers 1501 and 1511. Markers 1502 and 1512 indicate the location of slot 1502H by the space between the markers 1502 and 1512. Markers 1503 and 1513 indicate the location of slot 1503H by the space between the markers 1503 and 1513. In the example shown in FIG. 15A, the ablation-probe holes 1501H, 1502H, 1503H are sized so that a 17 gauge ablation probe or ablation probe introducer can pass through each hole. The guide-needle slot 1550 is sized to admit and align a 27-gauge spinal needle. The radius R is 10 mm. The angle A1 is 45 degress. The angle A2 is 45 degrees. The angle A3 is 75 degrees. The angle A4 is 90 degrees. The guideblock height H is 2 cm. In one example, such as shown in FIG. 15B, guideblock 1500 can be used to align up to three ablation probes, such as internally-cooled RF ablation probes, relative to each other and to the lateral aspect of a sacral foramina, into which a guide needle is inserted. For example, as shown by block 1500B in FIG. 15B, an ablation probe can be positioned using guideblock 1500 at each of the clock positions 2:30, 4:00, and 5:30 relative to a right sacral foramina. For example, as shown by block 1500A in FIG. 15B, an ablation probe can be positioned using guideblock 1500 at each of the clock positions 9:30, 8:00, and 6:30 relative to a left sacral foramina. In other embodiments, the dimensions H, W, A1, A2, A3, A4, R, and the slot widths can have different dimensions to suit clinical needs. In some other embodiments, guideblock can include additional image-guidance markers that indicate principle directions of the guideblock and its included slots. In some embodiments, the guide-needle slot 1520 is widened substantially, even to the point of removing all but a small amount of guideblock material around the holes 1501H, 1502H, 1503H and the markers Markers 1501, 1511, 1502, 1512, 1503, 1513; this has the advantage that the guide needle position at the skin surface does not interfere with the positioning of the guideblock 1500 if the guide needle is not aligned with the block 1500. In some embodiments, the markers 1511, 1512, 1513 can be omitted. In some embodiments, a circular marker, or segments thereof, can be included in guideblock 1500, for example centered on circle 1520.

Referring now to FIG. 15B, two guideblocks 1500A and 1500B of the kind shown in FIG. 15A are shown in process of aligning cooled RF ablation probes 1531, 1542 (and others) percutaneously relative to the lateral aspect of two sacral foramina S1 left 1591 and S2 right 1592 at which guide needles 1530 and 1540 having been placed, respectively; each ablation probe 1531, 1542 (and others) generating a monopolar RF heat lesion 1531L, 1542L (and others) that disrupts one or more sacral lateral branch nerves 1581, 1582 (and others) innervating the SIJs 1595 and 1596 of sacrum 1590, respectively. The guideblocks 1500A and 1500B are positioned on the dorsal skin surface and are identical to each other, except that they are positioned relative to a different guide needle, and are rotated around the guide needle relative to each other. The embodiments of a guideblock 1500A and 1500B each have the advantage that the cooled RF ablation probes are stabilized from falling over when positioned in the body when tissue overlaying the sacrum 1590 is thin. The embodiments of a guideblock 1500A and 1500B each have the advantage that they constraint the relative position of the RF ablation probes relative to each other and thereby ensure overlap between adjacent monopolar cooled RF heat lesions, which can be difficult to ensure if placing RF ablation probes at depth free-hand, perhaps only using a ruler. In some examples, non-cooled RF ablation electrodes can be used with guideblocks 1500A and 1500B. In some examples, only one guideblock 1500A is used to lesion around multiple sacral foramina by moving the guideblock 1500A from one foramina to another, typically the S1, S2, and S3 foramina on one side.

Referring to FIGS. 1 through 15, each of the guideblocks 100, 200, 300, 400, 500, 600, 700, 800, 900, 1000, 1100, 1200, 1300, 1400, and 1500 can further include one or more of the following: x-ray visible markers; CT-visible markers; MRI-visible markers; equally-spaced guide holes; unequally-spaced guide holes; inter-guidehole spacings that are larger for more peripheral guideholes than for more centrally located guideholes; additional guidehole to allow the user to adjust the spacing of ablation probes; image-guidance markers that are proximate to each of several guideholes; x-ray visible guideholes; CT-visible guideholes; MRI-visible guideholes; curved surfaces to conform to patient anatomy; a multiplicity of image-guidance markers for each guidehole; a reduced-weight cross-section; a T-shaped cross section; a shape configured to improve stability of the guideblock; shape configured to improve support for ablation probe inserted through the guideholes; non-parallel guideholes; an image-guidance marker that indicates the ultimate location of a part of an ablation probe, such as the distal end or the action tip, that is inserted into tissue to a depth through a guidehole; lateral openings for one or more guideholes; a clamp for the lateral opening for a guidehole; a non-linear arrangement of guideholes; an arrangement of guideholes that matches the geometry of target anatomy; a connection between the signal output of a HF ablation signal generator and an ablation probe inserted through the guideblock; guideholes arranged in polygonal arrays that are either parallel, non-parallel, or both; an inter-guidehole distance indicator. Each of the guideblocks in FIGS. 1-15 can be adjusted to have a number of guideholes that is at least two. The dimensions of each of the guideblocks in FIG. 1-15 can be adjusted to suit clinical needs, for example within the dimensional ranges for inter-guidehole spacing, probe diameter, block thickness, guidehole clearance and other dimensions described in relation to FIG. 1. Each of the guideblocks in FIGS. 1 through 15 can be adapted for use with needles, injection needles, injection electrodes, RF electrodes, RF cannula, cooled RF electrode systems, MW ablation antennae, non-cooled RF electrodes, stimulation electrodes, multi-electrode ablation systems, single-electrode ablation systems, single probe ablation systems, single-output ablation systems. Each of the guideblocks in FIGS. 1 through 15 can be adapted for use with monopolar, bipolar, multi-polar, and combinations and sequences thereof. Each of the guideblocks in FIGS. 1 through 15 can be used with ablation probes that produce heat lesions sequentially or at the same time. Guideblocks of the types shown in FIG. 1 through 15 can be used for tissue ablation in a wide variety of clinical contexts including tissue coagulation, pain management, tumor ablation, cardiac ablation, tissue devascularization, open surgical procedures, percutaneous surgical procedures, laparoscopic surgical procedures, facet denervation, SIJ deneravation, pulsed RF neuromodulation, pulsed RF lesioning, preparation of collapsed bone for injection of bone cement. Guideblocks of the types shown in FIG. 1 through 15 can be used for tissue ablation in all parts of the human body including the spine, bone, spinal nerve, peripheral nerve, knee nerve, hip nerve, shoulder nerve, foot nerve, hand nerve, carpel tunnel, sympathetic nerve, trigeminal nerve, medial branch nerve, sacral lateral branch nerve, brain, heart, liver, kidney, lung, pancreas, prostate, adrenal gland, thyroid, gall bladder, vertebral body, intervertebral nerve, basivertebral nerve, an intervertebral disc, nerve in an intervertebral disc, posterior annulus of an intervertebral disc, nucleus of the intervertebral disc, muscle, osteoid osteoma.

A number of embodiments of the invention have been described. Nevertheless, it will be understood that various modifications may be made without departing from the spirit and scope of the invention. Accordingly, other embodiments are within the scope of the following claims. What we claim are the following:

What is claimed is:

1. A guideblock for alignment of two or more high-frequency-tissue-ablation probes in a body.

2. The system of claim 1 wherein the guideblock includes slots that orients the ablation probes in one or more of the configurations selected from the group: all probes parallel, no two probes are parallel, some probes are parallel and some probes are not parallel, probes spaced for bipolar ablation, probes spaced for the production of overlapping bipolar lesions, probes spaced for bipolar RF ablation, probes spaced for bipolar RF ablation between the active tips of physically separate probes, probes spaced for generation of a singular bipolar RF lesion that fills in the space between two physically separate ablation probes, probes spaced for monopolar ablation, probes spaced for the generation of overlapping monopolar lesions, probes spaced for monopolar RF ablation, probes spaced for microwave ablation, probes spaced to form a cluster, probes oriented to avoid an anatomical structure, probes positioned to form a cluster within a target a first anatomical region or regions and to avoid an second anatomical region or regions, probes positioned to avoid one or more ribs and to position the probe active tips in a desired arrangement in a target anatomical region, probes positioned to avoid one or more ribs and to position the probe active tips in a cluster in the liver, probes positioned to avoid one or more ribs and to position the probe active tips in a cluster in the lung, probes positioned perpendicular to a bone surface, probes positioned parallel to a bone surface, proves positioned tangent to an anatomical surface, probes positions perpendicular to an anatomical surface, probes positioned in a row, probes positioned in a straight row, probes positioned in a curved row, probes positioned in a triangular array, probes positioned in an isolosceles triangle arrangement, probes positioned in a square array, probes positioned in a rectangular array, probes positioned in a parallel polygonal array, probes positioned in two parallel rows, probes positioned such that a first probe and second probe are parallel and third probe is not parallel to the first probe or second probe, probes perpendicular to the dorsal sacrum, probes positioned with distal points touching or almost touching the dorsal sacrum, probes positioned across space between the transverse processes of two adjacent thoracic vertebra, probes positioned perpendicular to the lateral articular pillar of a cervical vertebra, probes positioned tangent to the lateral articular pillar of a cervical vertebra, probes positioned tangent to the notch between the transverse process and the superior articular process of a lumbar vertebra, probes positioned perpendicular to the notch between the transverse process and the superior articular process of a lumbar vertebra, probes positioned in an intervertebral disc, probes positioned in the posterior annulus of an intervertebral disc, probes positioned along a the path of a desired cut, probes positioned in an organ in a desired arrangement, probes positions in a bone in a desired arrangement, probes positioned in a vertebra, probes positioned across the basivertebral nerve in a vertebra, probes position within or across an osteoid osteoma.

3. The system of claim 1 wherein the guideblock includes one or more markers for image guidance of the guideblock's position relative to the body.

4. The system of claim 3 wherein a marker is visible in one or more of the medical image modalities selected from the group: x-ray, fluoroscopy x-ray, CT, MRI, PET.

5. The system of claim 3 wherein the guideblock includes one or more slots, and one or more markers that indicates the position of each slot for guidance of an ablation probe.

6. The system of claim 3 wherein a marker indicates the bodily position of the active tip of an ablation probe inserted through a slot to a predetermined depth.

7. The system of claim 1 that further includes a depth stop for one or more ablation probe.

8. The system of claim 1 wherein each ablation probe is of one or more of the types selected from the group: radiofrequency cannula, radiofrequency electrode, radiofrequency injection electrode, monopolar radiofrequency, bipolar radiofrequency, multi-contact radiofrequency electrode, cooled RF, impedance-controlled cooled RF, temperature-controlled cooled RF, cooled RF electrode with extension tip thermosensor, cooled radiofrequency electrode with movable thermosensor positioned distal to the active tip, radiofrequency electrode controlled using a satellite thermosensor that is physically separate from the radiofrequency electrode, cooled radiofrequency electrode controlled using a satellite thermosensor that is physically separate from the radiofrequency electrode, internally-cooled radiofrequency electrode, fluid-cooled RF electrode, perfusion radiofrequency electrode, bipolar cooled RF, internally-cooled electrode, perfusion electrode, microwave, microwave antenna, internally-cooled microwave electrode, direct current, high-frequency current, high-frequent stimulation nerve block signal, stimulation, neurolytic injection needle, injection needles, anesthetic injection needle.

9. The system of claim 1 wherein the guideblock is sized to stabilize the portion of the ablation probe outside the body.

10. The system of claim 1 wherein guideblock is configured for one or more of the clinical objectives selected from the group: tumor ablation, partial tumor ablation, tissue ablation, tissue coagulation, tissue devascularization, tissue devascularization for bloodless resection, an open surgical procedure, percutaneous tissue ablation, laparocopic ablation nerve ablation, brain ablation, ablation of the intervertebral disc, ablation of a bone, ablation of a vertebra, ablation of osteoid osteoma, pain management, cancer therapy, movement disorder treatment, neurosurgery, surgical tumor resection, surgical tissue resection, ablation in the liver ablation, ablation in the lung, ablation in the pancreas, ablation in the kidney, ablation in the adrenal gland, ablation in the thyroid, SIJ denervation, ablation of some or all of the dorsal sacral innervation, ablation of the sacral lateral branch nerves, ablation of the lumbar L5 dorsal ramus nerve or branch thereof, ablation of a lumbar medial branch nerve, ablation of a thoracic medial branch nerve, ablation of a cervical medial branch nerve, facet denervation, ablation of a peripheral nerve, ablation of the sympathetic chain.

11. The system of claim 1 wherein the guideblock includes a multiplicity of slots that provide for physician selection of the relative position of ablation probes.

12. The system of claim 11 wherein the guideblock further includes an indicator of the relative spacing of the slots.

13. The system of claim 1 wherein the guideblock includes two or more slots for positioning of ablation probes within the body, wherein one or more slots includes an opening for lateral separation of the guideblock and an ablation probe inserted through the slot.

14. The system of claim 13 wherein the guideblock further includes one or more clamps for closing off one or more of the openings.

15. The system of claim 1 wherein the electrical ablation signal output delivered to at least one ablation probe aligned by the guideblock is controlled in whole or in part by a temperature measured by at least one temperature probe aligned by the guideblock in the body.

16. The system of claim 1 wherein the guideblock includes a row of slots for placement of a row of radiofrequency ablation probes at or near the dorsal surface of the sacrum, wherein the slots orient the radiofrequency ablation probes so that the probes are parallel to each other, and the slots space the probes for generation of bipolar radiofrequency heat lesions between each adjacent pair of radiofrequency ablation probes.

17. The system of claim 1 wherein the guideblock includes three slots for alignment of three internally-cooled RF electrodes around the lateral aspect of a sacral foramina.

18. A method for tissue ablation comprising: orienting a guideblock to a body, inserting two or more ablation probes into the body through the guideblock, ablating tissue using the ablation probes.

19. A method of placement of two or more ablation probes in a geometric arrangement within the living body comprising: imaging a guideblock in relation to a body, determining a target location in the body for each ablation probe by means of the image of a marker visible in the image and having a geometric relationship to slots in the guideblock, inserting each ablation probe through a slot in the guideblock into the body, ablating tissue using the ablation probes.

20. The method of claim 19 and further comprising: removing all ablation probes except one remaining ablation probe, repositioning the guideblock relative to the one remaining ablation probe, re-inserting one or more ablation probes through a slot in the guideblock into the body, ablating tissue using the ablation probes.
